(19) United States
(12) Patent Application Publication
(10) Pub. No.: US 2026/0139325 A1
(43) Pub. Date: May 21, 2026

(54) CLASSIFICATION OF COLORECTAL TUMORS USING DNA METHYLATION FROM LIQUID BIOPSY

(71) Applicant: GUARDANT HEALTH, INC., Palo Alto, CA (US)

(72) Inventor: Catalin BARBACIORU, Fremont, CA (US)

(21) Appl. No.: 19/446,794

(22) Filed: Jan. 12, 2026

Related U.S. Application Data
(63) Continuation of application No. PCT/US2024/037754, filed on Jul. 12, 2024.
(60) Provisional application No. 63/513,666, filed on Jul. 14, 2023.

(51) Int. Cl. C12Q 1/6886

(52) U.S. Cl. CPC C12Q 1/6886; C12Q 2600/154; C12Q 2600/158; G01N 2800/50

(57) ABSTRACT

Described herein are gene signatures providing prognostic, diagnostic, treatment and molecular subtype classifications of cancers through genomic and epigenomic profiling, Methods and compositions for determining cancer and subtypes, including breast cancer are described and specific and sensitive detection of biomarkers of interest is provided. Such biomarkers are indicative of disease pathogenesis, which provides opportunity for selection of treatment, including treatment regimes directed at identifying candidates for responsiveness and overcoming resistance mechanisms.

### CROSS-REFERENCE TO RELATED APPLICATIONS

The present application is a Continuation of International Patent Application No. PCT/US2024/037754, filed Jul. 12, 2024, which claims the benefit of priority of U.S. Provisional Patent Application No. 63/513,666, filed Jul. 14, 2023, which is incorporated by reference herein in its entirety for all purposes.

### BACKGROUND

Cancer is a major cause of disease worldwide. Each year, tens of millions of people are diagnosed with cancer around the world, and more than half of the patients eventually die from it. In many countries, cancer ranks the second most common cause of death following cardiovascular diseases. Early detection is associated with improved outcomes for many cancers.

To detect cancer, several screening tests are available. A physical exam and history survey general signs of health, including checking for signs of disease, such as lumps or other unusual physical symptoms. A history of a patient's health habits and past illnesses and treatments will also be taken. Laboratory tests are another type of screening test and may include medical procedures to procure samples of tissue, blood, urine, or other substances in the body before conducting laboratory testing. Imaging procedures screen for cancer by generating visual representations of areas inside the body. Genetic tests detect certain gene deleterious mutations linked to some types of cancer. Genetic testing is particularly useful for a number of diagnostic methods.

Tumor-specific DNA methylation patterns are well-described in human malignancies. The potential existence of the ‘CpG island methylator phenotype’ (CIMP) in colorectal cancer has been observed in tissue studies where CIMP subtypes were associated with molecular and clinical features. One example includes Guinney et al. Nature Medicine (2015), which describes consensus molecular subtypes based on a composite of gene expression data, and Fennell CMGH (2019). There is a great need in the art to identify CIMP subtypes using a methylation-based liquid biopsy profiling system. consuming as relying on tumor tissue and visual inspection using immunohistochemial staining.

Described herein is a method of the characterization of CIMP profiles for CRC patients using a convenient and non-invasive liquid biopsy approach. As demonstrated herein, validation of liquid biopsy-based CIMP profiling of these tumor indications is shown as concordant with tissue, without associated difficulties of tissue biopsy.

### SUMMARY OF THE INVENTION

Described herein is a method, comprising detecting methylation in at least one of a plurality of sites; generating a plurality of one or more metrics for each of the plurality of sites; and processing the one or more metrics to characterize a sample. In various embodiments, the one or more metrics are obtained from methylation calls from each of the plurality of sites. In various embodiments, the method includes obtaining a sample. In various embodiments, the method includes having obtained a sample. In various embodiments, the cancer is colon cancer. In various embodiments, the method includes building a classification model from methylation data of a set of training samples comprising one or more of: consensus molecular subtypes (CMS)1, CMS2, CMS3 and CMS4. In various embodiments, the cancer is colon cancer. .In various embodiments, the classification model is trained using clustering. In various embodiments, the method includes training includes ranking. In various embodiments, the regions are selected using based on ranking. In various embodiments, the method includes ranking based on significance and/or weights for each of the plurality of sites. In various embodiments, the classification model is trained using cross-validation. In various embodiments, the method includes using 1, 2, 3, 4, 5, 6, 7, 8, 9, 10-fold or more cross-validation. In various embodiments, a plurality of one or more metrics for each of the plurality of sites includes a linear regression, Bernoulli probability distribution and/or a mixture model. In various embodiments, the one or more metrics to characterize a sample includes includes calculating the probability of tumor molecules based on an amount of input DNA in one or more of the plurality of sites. In various embodiments, the sites include a custom panel. In various embodiments, the method includes custom panel is configured in an in silico panel. In various embodiments, the custom panel is configured in a physical panel. In various embodiments, the custom panel includes a set of oncogenes, promoter regions for a set of oncogenes, HRR genes, immuno-oncology (IO) genes, a cancer pathway, methylation peaks found in cancer or methylation peaks found in clinical samples. In various embodiments, the method includes custom panel is refined based at least on literature annotations, common methylation peak positions, and/or public datasets. In various embodiments, the plurality of sites include one or more genes selected from the group consisting of: NEFM, SOX4, INTS1, RPSAP52,HMGA2, TMEM217,TBC1D22B, ADGRB1, AFAP1L2, LOC101927179,CORIN, MRPL4, FBXO24, BBC3, RNF167, RGMB, ZFYVE16, TRIM39,HCG18, ERICD, COPRS, MAFK, CNTNAP2, SHH, NDUFB9, LVRN, GAD2, MYL6B, NFXL1, RAD51, DNAJC25-GNG10, LINC00693, SOX4, PDE1B, MYC, KHDRBS2, RARRES2, LINC02062,CHD1, ARL13B, ZBTB18, H4C3, LOC390877, CCDC184, SUSD6, ABR, TUBGCP3, OTX1, PLEKHD1, SOX2-OT, ARL2-SNX15,ARL2, GFI1, C5orf38, LDB3, ERFE, KCNK10, TMEM145, SPTBN2, PLCG2, CYSTM1, C1orf21, HPCAL1, DGLUCY, ERICD, AGO3, RASA1, AHDC1, P2RY2, MIR9-3HG, TENT5A, FA2H, NUCKS1, SSH2, SCRN3, LOC344967,N4BP2, PALM2AKAP2, TRAF1, NUCKS1, YPEL5, HAND2-AS1, SLC25A37, WIPF3, CFAP221, RAD21-AS1,RAD21, PPFIA3, ADRA1A, KHSRP, TRIM39-RPP21,RPP21, MAPRE2, LOC344967, N4BP2, LOC115308161,ARID1B, STYX, PCDHGA2, PCDHGA12, PCDHGA9, PCDHGB7, PCDHGA3,PCDHGA10,PCDHGC3,PCDHGA5,PCDHGA6,PCDHGB4,PCDHGA7,PCDHGB, PCDHGA1,PCDHGB1,PCDHGA4,PCDHGB2,PCDHGB3,PCDHGA11,PCDHGB5,PCDHGA8, LRAT, JARID2-AS1,JARID2, TNS3, FOLH1, STX1A, H4C3, SEMA3F, SEMA3F-AS1, NDRG4, POLE, IHH, LOC388780, RPL23, CUX1, LNX2, PTPN2, RTN4RL2, SLC30A1, LOC100129917, CTD-2297D10.2, RUSC1,RUSC1-AS1, YLPM1, GRIK3, MAD1L1, RAG1, MARCHF3, NEXN-AS1,NEXN, EXD3, PYCR1, ICAM5, ERFL, SMAD7, FAM219A, CDCA7, LOC388780, LOC100505715, CD58, CAMTA2, HS3ST4, CHD3, PRNP, FAM189B, TTC9C, ITPKB, FRMD4A, DENND4B, MCIDAS, TXNL4A, IDI1, KCNH2, SHC1, LOC101928120, AGTRAP, MSC-AS1,MSC, WBP1,INO80B-WBP1, EEF1AKMT4, EEF1AKMT4-ECE2. In various embodiments, the method includes one or more genes selected from the group consisting of: MYC, BBC3,and RAG1. In various embodiments, the method includes characterizing the sample by determining gene expression of one or more biomarkers.

Described herein is a method comprising: detecting methylation in at least one of a plurality of sites; generating a plurality of methylation calls for each of the plurality of sites; obtaining one or more metrics from the methylation calls; and processing the one or more metrics to generate a probability that a patient is afflicted with cancer. In various embodiments, the method includes generating a probability that a patient is afflicted with cancer includes determination of a cancer subtype. In various embodiments, the cancer subtype is characterized as one or more of more of consensus molecular subtypes (CMS)1, CMS2, CMS3 and CMS4. In various embodiments, the cancer is colon cancer. In various embodiments, the plurality of sites include one or more genes selected from the group consisting of: NEFM, SOX4, INTS1, RPSAP52,HMGA2, TMEM217,TBC1D22B, ADGRB1, AFAP1L2, LOC101927179,CORIN, MRPL4, FBXO24, BBC3, RNF167, RGMB, ZFYVE16, TRIM39,HCG18, ERICD, COPRS, MAFK, CNTNAP2, SHH, NDUFB9, LVRN, GAD2, MYL6B, NFXL1, RAD51, DNAJC25-GNG10, LINC00693, SOX4, PDE1B, MYC, KHDRBS2, RARRES2, LINC02062,CHD1, ARL13B, ZBTB18, H4C3, LOC390877, CCDC184, SUSD6, ABR, TUBGCP3, OTX1, PLEKHD1, SOX2-OT, ARL2-SNX15,ARL2, GFI1, C5orf38, LDB3, ERFE, KCNK10, TMEM145, SPTBN2, PLCG2, CYSTM1, C1orf21, HPCAL1, DGLUCY, ERICD, AGO3, RASA1, AHDC1, P2RY2, MIR9-3HG, TENT5A, FA2H, NUCKS1, SSH2, SCRN3, LOC344967,N4BP2, PALM2AKAP2, TRAF1, NUCKS1, YPEL5, HAND2-AS1, SLC25A37, WIPF3, CFAP221, RAD21-AS1,RAD21, PPFIA3, ADRA1A, KHSRP, TRIM39-RPP21,RPP21, MAPRE2, LOC344967, N4BP2, LOC115308161,ARID1B, STYX, PCDHGA2, PCDHGA12, PCDHGA9, PCDHGB7, PCDHGA3, PCDHGA10, PCDHGC3, PCDHGA5, PCDHGA6, PCDHGB4,PCDHGA7,PCDHGB6,PCDHGA1,PCDHGB1,PCDHGA4,PCDHGB2,PCDHGB3, PCDHGA11,PCDHGB5,PCDHGA8, LRAT, JARID2-AS1,JARID2, TNS3, FOLH1, STX1A, H4C3, SEMA3F, SEMA3F-AS1, NDRG4, POLE, IHH, LOC388780, RPL23, CUX1, LNX2, PTPN2, RTN4RL2, SLC30A1, LOC100129917, CTD-2297D10.2, RUSC1,RUSC1-AS1, YLPM1, GRIK3, MAD1L1, RAG1, MARCHF3, NEXN-AS1,NEXN, EXD3, PYCR1, ICAM5, ERFL, SMAD7, FAM219A, CDCA7, LOC388780, LOC100505715, CD58, CAMTA2, HS3ST4, CHD3, PRNP, FAM189B, TTC9C, ITPKB, FRMD4A, DENND4B, MCIDAS, TXNL4A, IDI1, KCNH2, SHC1, LOC101928120, AGTRAP, MSC-AS1,MSC, WBP1,INO80B-WBP1, EEF1AKMT4, EEF1AKMT4-ECE2. In various embodiments, the plurality of sites include one or more genes selected from the group consisting of: MYC, BBC3,and RAG1. In various embodiments, the method includes selecting a treatment is selected from bevacizumab, cetuximab, encorafenib, ipilimumab, nivolumab, panitumumab, pembrolizumab, ramucirumab, regorafenib, tucatinib, ziv-aflibercept, adagrasib, and/or fruquintinib. In various embodiments, the method includes administering a treatment to the patient, wherein the treatment is bevacizumab, cetuximab, encorafenib, ipilimumab, nivolumab, panitumumab, pembrolizumab, ramucirumab, regorafenib, tucatinib, ziv-aflibercept, adagrasib, and/or fruquintinib.

Described herein is a method of determining a diagnosis of, prognosis of, susceptibility to, cancer in an individual, comprising: determining the presence or absence of a lower or higher level of expression in the individual relative to a normal baseline standard for a gene. In various embodiments, the genes include a E2F factor pathway target, including E2F1. In various embodiments, the genes include one or more of: MYC, BBC3, RAG1. In other embodiments, the method includes cancer subtyping, such as consensus molecular subtypes (CMS)1, CMS2, CMS3 and CMS4. In various embodiments, the cancer is colon cancer. In other embodiments, the method includes selecting a treatment for a subject. In other embodiments, the method includes administering a treatment for a subject. In various embodiments, the treatment is selected from bevacizumab, cetuximab, encorafenib, ipilimumab, nivolumab, panitumumab, pembrolizumab, ramucirumab, regorafenib, tucatinib, ziv-aflibercept, adagrasib, and/or fruquintinib

For example, disclosed is a method of selecting a therapeutic treatment for a subject, comprising: a) obtaining a biological sample from the subject; b) determining the expression levels of MYC, BBC3, RAG1 in the biological sample; c) comparing the expression levels to a control; and d) selecting a therapeutic treatment based on the comparison. In some embodiments, the control is from one or more subjects which do not have cancer. In some embodiments, the expression levels are inferred from the analysis of cfDNA. In some embodiments, the abnormal expression of MYC, BBC3, RAG1 indicates that the subject is resistant to a therapeutic treatment, and therefore an alternative therapeutic treatment is selected. In some embodiments, the method involves administering the alternative therapy. In some embodiments, the method involves determining the expression of MYC, BBC3, RAG1. In other embodiments, the method includes cancer subtyping, such as consensus molecular subtypes (CMS)1, CMS2, CMS3 and CMS4. In various embodiments, the cancer is colon cancer.

Also disclosed is a method of selecting a therapeutic treatment for a subject, comprising: a) obtaining a biological sample from the subject; b) determining the expression levels of one or more components of the a E2F factor pathway target, including E2F1, in the biological sample; c) comparing the expression levels to a control; and d) selecting a therapeutic treatment based on the comparison. In some embodiments, the control is from one or more subjects which do not have cancer. In some embodiments, the expression levels are inferred from the analysis of cfDNA. In some embodiments, the abnormal expression of the one or more components of the a E2F factor pathway target, including E2F1, pathway indicates that the subject is resistant to a therapeutic treatment, and therefore an alternative therapeutic treatment is selected. In some embodiments, the method involves administering the alternative therapy.

Also disclosed is a method of identifying whether a subject is resistant to a therapeutic treatment, wherein the method includes: a) obtaining a biological sample from the subject; b) determining the expression levels of one or more components of the a E2F factor pathway target, including E2F1, pathway in the biological sample; c) comparing the expression levels to a control; and d) classifying the subject as resistant to the therapeutic treatment based on the comparison. In some embodiments, the control is from one or more subjects which do not have cancer. In some embodiments, the expression levels are inferred from the analysis of cfDNA. In some embodiments, the abnormal expression of the one or more components of the a E2F factor pathway target, including E2F1, pathway indicates that the subject is resistant to a therapeutic treatment. In some embodiments, the method involves administering an alternative therapy.

Also disclosed is the use of one or more components of a E2F factor pathway target, including E2F1, pathway as a biomarker for cancer. In some embodiments, the biomarker is for resistance to a therapeutic treatment for cancer. In other embodiments, the method includes cancer subtyping, such as consensus molecular subtypes (CMS)1, CMS2, CMS3 and CMS4. In various embodiments, the cancer is colon cancer.

In some embodiments, the results of the systems and methods disclosed herein are used as an input to generate a report. The report may be in a paper or electronic format. For example, genetic results as determined by the methods and systems disclosed herein, such as the presence of a nucleic acid variant was detected in a sample, can be displayed directly in such a report. In some embodiments, only the presence or absence of a disease, such as cancer, is displayed in such a report.

The various steps of the methods disclosed herein, or steps carried out by the systems disclosed herein, may be carried out at the same or different times, in the same or different geographical locations, e.g., countries, and/or by the same or different people. In some embodiments, the report is communicated to a subject, for example, a subject who has cancer and has undergone testing by the methods and systems described herein, or to a healthcare professional, such as a physician treating the subject that has cancer.

### BRIEF DESCRIPTION OF FIGURES

FIG. 1. Samples and data.

FIG. 2. CNV data (SCNA sample scoring).

FIG. 3. CNV data (recurrent focal amplifications).

FIG. 4. Methylation data hierarchical clustering.

FIG. 5. cfDNA methylation data hierarchical clustering (2 clusters).

FIG. 6. TCGA tissue methylation data, same target regions.

FIG. 7. cfDNA methylation data hierarchical clustering.

FIG. 8. cfDNA methylation data hierarchical clustering (4 clusters)

FIG. 9. CRC subtype predictor genomic regions were used to perform gene set enrichment analyses, the barplot shows significantly enriched transcription factors and their corresponding multiple testing adjusted p-values.

FIG. 10. Shows E2F1 target genes MYC, BBC3,RAG1 are colorectal cancer genes (adjusted p-value=0.002187) with functions across multiple cancer pathways including P53, ErbB, and TGF-Beta signaling pathways.

FIG. 11 Shows transcription factor enrichment for the CRC predictor genomic regions and their corresponding multiple testing adjusted p-values.

### DETAILED DESCRIPTION

While various embodiments of the disclosure have been shown and described herein, those skilled in the art will understand that such embodiments are provided by way of example only. Numerous variations, changes, and substitutions may occur to those skilled in the art without departing from the disclosure. It should be understood that various alternatives to the embodiments of the disclosure described herein may be employed.

The term “about” and its grammatical equivalents in relation to a reference numerical value can include a range of values up to plus or minus 10% from that value. For example, the amount “about 10” can include amounts from 9 to 11. The term “about” in relation to a reference numerical value can include a range of values plus or minus 10%, 9%, 8%, 7%, 6%, 5%, 4%, 3%, 2%, or 1% from that value.

The term “at least” and its grammatical equivalents in relation to a reference numerical value can include the reference numerical value and greater than that value. For example, the amount “at least 10” can include the value 10 and any numerical value above 10, such as 11, 100, and 1,000.

The term “at most” and its grammatical equivalents in relation to a reference numerical value can include the reference numerical value and less than that value. For example, the amount “at most 10” can include the value 10 and any numerical value under 10, such as 9, 8, 5, 1, 0.5, and 0.1.

As used herein the singular forms “a”, “an”, and “the” can include plural referents unless the context clearly dictates otherwise. Thus, for example, reference to “a cell” can include a plurality of such cells and reference to “the culture” can include reference to one or more cultures and equivalents thereof known to those skilled in the art, and so forth. All technical and scientific terms used herein can have the same meaning as commonly understood to one of ordinary skill in the art to which this disclosure belongs unless clearly indicated otherwise.

Cancer can be indicated by epigenetic variations, such as methylation. Examples of methylation changes in cancer include local gains of DNA methylation in the CpG islands at the transcription start site (TSS) of genes involved in normal growth control, DNA repair, cell cycle regulation, and/or cell differentiation. This hypermethylation can be associated with an aberrant loss of transcriptional capacity of involved genes and occurs at least as frequently as point mutations and deletions as a cause of altered gene expression. DNA methylation profiling can be used to detect regions with different extents of methylation (“differentially methylated regions” or “DMRs”) of the genome that are altered during development or that are perturbed by disease, for example, cancer or any cancer-associated disease. The genome of cancer cells harbor imbalance in the above DNA methylation patterns, and therefore in functional packaging of the DNA. The abnormalities of chromatin organization are therefore coupled with methylation changes and may contribute to enhanced cancer profiling when analyzed jointly. Combining MBD-partitioning with fragmentomic data, such as fragment mapped starts and stops positions (correlated with nucleosome positions), fragment length and associated nucleosome occupancy, can be used for chromatin structure analysis in hypermethylation studies with the aim to improve biomarker detection rate.

Methylation profiling can involve determining methylation patterns across different regions of the genome. For example, after partitioning molecules based on extent of methylation (e.g., relative number of methylated sites per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

A characteristic of nucleic acid molecules may be a modification, which may include various chemical or protein modifications (i.e. epigenetic modifications). Non-limiting examples of chemical modification may include, but are not limited to, covalent DNA modifications, including DNA methylation. In some embodiments, DNA methylation includes addition of a methyl group to a cytosine at a CpG site (a cytosine followed by a guanine in a nucleic acid sequence). In some embodiments, DNA methylation includes addition of a methyl group to adenine, such as in N6-methyladenine. In some embodiments, DNA methylation is 5-methylation (modification of the 5th carbon of the 6 carbon ring of cytosine). In some embodiments, 5-methylation includes addition of a methyl group to the 5C position of the cytosine to create 5-methylcytosine (m5c). In some embodiments, methylation includes a derivative of m5c. Derivatives of m5c include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6 carbon ring of cytosine). In some embodiments, 3C methylation includes addition of a methyl group to the 3C position of the cytosine to generate 3-methylcytosine (3mC). Other examples include N6-methyladenine or glycosylation. DNA methylation includes addition of methyl groups to DNA (e.g. CpG) and can change the expression of methylated DNA region.. Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

Specifically, as one example, colorectal cancer is a heterogeneous disease characterized by distinct genetic and epigenetic changes that drive proliferative activity and inhibit apoptosis with a conventional pathway to colorectal cancer as involving APC mutation and chromosomal instability. This conventional pathway accounts for a majority, approximately 75%, of sporadic cancers. The remaining colorectal cancers arise from serrated polyps and have activating mutations in the BRAF proto-oncogene, frequent microsatellite instability (MSI), and the CpG island methylator phenotype (CIMP). Differential CpG island and shore hypermethylation are frequently observed methylation events in cancer, with probes on the north and south CpG shelves, as well as those in the open seas, being reported as frequently hypomethylated across most cancers. The implications of hypomethylated CpG dinucleotides outside of CpG islands are unclear.

A CpG dyad is the dinucleotide CpG (cytosine-phosphate-guanine, i.e. a cytosine followed by a guanine in a 5′→3′ direction of the nucleic acid sequence) on the sense strand and its complementary CpG on the antisense strand of a double-stranded DNA molecule. CpG dyads can be either fully methylated or hemi-methylated (methylated on one strand only).

The CpG dinucleotide is underrepresented in the normal human genome, with the majority of CpG dinucleotide sequences being transcriptionally inert (e.g. DNA heterochromatic regions in pericentromeric parts of the chromosome and in repeat elements) and methylated. However, many CpG islands are protected from such methylation especially around transcription start sites (TSS).

Protein modifications include binding to components of chromatin, particularly histones including modified forms thereof, and binding to other proteins, such as proteins involved in replication or transcription. The disclosure provides methods of processing and analyzing nucleic acids with different extents of modification, such that the nature of their original modification is correlated with a nucleic acid tag and can be decoded by sequencing the tag when nucleic acids are analyzed. Genetic variation of sample nucleic acid modifications can then be associated with the extent of modification (epigenetic variation) of that nucleic acid in the original sample. include single stranded (e.g., ssDNA or RNA) or double stranded molecules (e.g., dsDNA).

The loss of DNA can reduce the presence of one or more types of DNA such that the presence of the one or more types of DNA such as cfDNA, is difficult to detect. In one or more additional scenarios, existing methods to measure DNA methylation, such as enrichment or depletion methods, can have a relatively high level of resolution, such as about 100 base pairs (bp) to about 200 bp that can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determinations of tumor fraction estimates can impact diagnosis and/or treatment decisions for individuals.

### Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids. A sample can be isolated or obtained from a subject and transported to a site of sample analysis. The sample may be preserved and shipped at a desirable temperature, e.g., room temperature, 4° C., −20°C., and/or −80° C. A sample can be isolated or obtained from a subject at the site of the sample analysis. The subject can be a human, a mammal, an animal, a companion animal, a service animal, or a pet. The subject may have a cancer. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed of being susceptible to cancer or any cancer-associated genetic mutations/disorders.

The volume of plasma can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000(104 ) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample includes an epigenetic variant associated with the presence of a genetic variant, wherein the sample does not comprise the genetic variant.

Exemplary amounts of cell-free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as Cot-1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

### Analytes

Analytes can include nucleic acid analytes, and non-nucleic acid analytes. The disclosure provides for detecting genetic variations in biological samples from a subject. Biological samples may include polynucleotides from cancer cells. Polynucleotides may be DNA (e.g., genomic DNA, cDNA), RNA (e.g., mRNA, small RNAs), or any combination thereof. Biological samples may include tumor tissue, e.g., from a biopsy. In some cases, biological samples may include blood or saliva. In particular cases, biological samples may comprise cell free DNA (“cfDNA”) or circulating tumor DNA (“ctDNA”). Cell free DNA can be present in, e.g., blood.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquity lati on variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. This further includes receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In general, the systems, apparatus, methods, and compositions can be used to analyze any number of analytes, further including both nucleic acid analytes and non-nucleic acid analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

One or more nucleic acid analytes and/or non-nucleic acid analytes constitute a set of molecular interactions in a biological system under study (e.g., cells), which may be regarded as “interactome”—the molecular interactions that occur between molecules belonging to different biochemical families (proteins, nucleic acids, lipids, carbohydrates, etc.) and also within a given family. In various embodiments, an interactome is a protein-DNA interactome (network formed by transcription factors (and DNA or chromatin regulatory proteins) and their target genes. In other embodiments, interactome refers to protein-protein interaction network(PPI), or protein interaction network (PIN). The methods described herein allow for study and analysis of the interactome. Techniques such as proteogenomics (whole genome sequencing, whole exome sequencing and RNA-seq, and mass spectrometry as examples) can support study of the interactome.

### Analysis

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic and other analyte data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

The present analyses are also useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens is changing during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and mutation analyses alone or in combination.

The present methods can be used to diagnose, prognose, monitor or observe cancers. or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### Determination of 5-Methylcytosine Pattern of Nucleic Acids

Bisulfite-based sequencing and variants thereof provides a means of determining the methylation pattern of a nucleic acid. In some embodiments, determining the methylation pattern includes distinguishing 5-methylcytosine (5mC) from non-methylated cytosine. In some embodiments, determining methylation pattern includes distinguishing N6-methyladenine from non-methylated adenine. In some embodiments, determining the methylation pattern includes distinguishing 5-hydroxymethylcytosine (5hmC), 5-formylcytosine (5fC), and 5-carboxylcytosine (5caC) from non-methylated cytosine. Examples of bisulfite sequencing include, but are not limited to oxidative bisulfite sequencing (OX-BS-seq), Tet-assisted bisulfite sequencing (TAB-seq), and reduced bisulfite sequencing (redBS-seq).

Oxidative bisulfite sequencing (OX-BS-seq) is used to distinguish between 5mC and 5hmC, by first converting the 5hmC to 5fC, and then proceeding with bisulfite sequencing as previously described. Tet-assisted bisulfite sequencing (TAB-seq) can also be used to distinguish 5mc and 5hmC. In TAB-seq, 5hmC is protected by glucosylation. A Tet enzyme is then used to convert 5mC to 5caC before proceeding with bisulfite sequencing, as previously described. Reduced bisulfite sequencing is used to distinguish 5fC from modified cytosines.

Generally, in bisulfite sequencing, a nucleic acid sample is divided into two aliquots and one aliquot is treated with bisulfite. The bisulfite converts native cytosine and certain modified cytosine nucleotides (e.g. 5-formylcytosine or 5-carboxylcytosine) to uracil whereas other modified cytosines (e.g., 5-methylcytosine, 5-hydroxylmethylcystosine) are not converted. Comparison of nucleic acid sequences of molecules from the two aliquots indicates which cytosines were and were not converted to uracils. Consequently, cytosines which were and were not modified can be determined. The initial splitting of the sample into two aliquots is disadvantageous for samples containing only small amounts of nucleic acids, and/or composed of heterogeneous cell/tissue origins such as bodily fluids containing cell-free DNA.

The present disclosure provides methods allowing bisulfite sequencing and variants thereof. These methods work by linking nucleic acids in a population to a capture moiety, i.e., a label that can be captured or immobilized. Capture moieties include, without limitation, biotin, avidin, streptavidin, a nucleic acid including a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase. Following linking of capture moieties to sample nucleic acids, the sample nucleic acids serve as templates for amplification. Following amplification, the original templates remain linked to the capture moieties but amplicons are not linked to capture moieties.

The capture moiety can be linked to sample nucleic acids as a component of an adapter, which may also provide amplification and/or sequencing primer binding sites. In some methods, sample nucleic acids are linked to adapters at both ends, with both adapters bearing a capture moiety. Preferably any cytosine residues in the adapters are modified, such as by 5methylcytosine, to protect against the action of bisulfite. In some instances, the capture moieties are linked to the original templates by a cleavable linkage (e.g., photocleavable desthiobiotin-TEG or uracil residues cleavable with USER™ enzyme, Chem. Commun. (Camb). 2015 Feb. 21; 51(15): 3266-3269), in which case the capture moieties can, if desired, be removed.

The amplicons are denatured and contacted with an affinity reagent for the capture tag. Original templates bind to the affinity reagent whereas nucleic acid molecules resulting from amplification do not. Thus, the original templates can be separated from nucleic acid molecules resulting from amplification.

Following separation or partition, the respective populations of nucleic acids (i.e., original templates and amplification products) can be subjected to bisulfite treatment with the original template population receiving bisulfite treatment and the amplification products not. Alternatively, the amplification products can be subjected to bisulfite treatment and the original template population not. Following such treatment, the respective populations can be amplified (which in the case of the original template population converts uracils to thymines). The populations can also be subjected to biotin probe hybridization for enrichment. The respective populations are then analyzed and sequences compared to determine which cytosines were 5-methylated (or 5-hydroxylmethylated) in the original. Detection of a T nucleotide in the template population (corresponding to an unmethylated cytosine converted to uracil) and a C nucleotide at the corresponding position of the amplified population indicates an unmodified C. The presence of C's at corresponding positions of the original template and amplified populations indicates a modified C in the original sample.

In some embodiments, a method uses sequential DNA-seq and bisulfite-seq (BIS-seq) NGS library preparation of molecular tagged DNA libraries. This process is performed by labeling of adapters (e.g., biotin), DNA-seq amplification of whole library, parent molecule recovery (e.g. streptavidin bead pull down), bisulfite conversion and BIS-seq. In some embodiments, the method identifies 5-methylcytosine with single-base resolution, through sequential NGS-preparative amplification of parent library molecules with and without bisulfite treatment. This can be achieved by modifying the 5-methyl-ated NGS-adapters (directional adapters; Y-shaped/forked with 5-methylcytosine replacing) used in BIS-seq with a label (e.g., biotin) on one of the two adapter strands. Sample DNA molecules are adapter ligated, and amplified (e.g., by PCR). As only the parent molecules will have a labeled adapter end, they can be selectively recovered from their amplified progeny by label-specific capture methods (e.g., streptavidin-magnetic beads). As the parent molecules retain 5-methylation marks, bisulfite conversion on the captured library will yield single-base resolution 5-methylation status upon BIS-seq, retaining molecular information to corresponding DNA-seq. In some embodiments, the bisulfite treated library can be combined with a non-treated library prior to enrichment/NGS by addition of a sample tag DNA sequence in standard multiplexed NGS workflow. As with BIS-seq workflows, bioinformatics analysis can be carried out for genomic alignment and 5-methylated base identification. In sum, this method provides the ability to selectively recover the parent, ligated molecules, carrying 5-methylcytosine marks, after library amplification, thereby allowing for parallel processing for bisulfite converted DNA. This overcomes the destructive nature of bisulfite treatment on the quality/sensitivity of the DNA-seq information extracted from a workflow. With this method, the recovered ligated, parent DNA molecules (via labeled adapters) allow amplification of the complete DNA library and parallel application of treatments that elicit epigenetic DNA modifications. The present disclosure discusses the use of BIS-seq methods to identify cytosine5-methylation (5-methylcytosine), but this should is not limiting. Variants of BIS-seq have been developed to identify hydroxymethylated cytosines (5hmC; OX-BS-seq, TAB-seq), formylcytosine (5fC; redBS-seq) and carboxylcytosines. These methodologies can be implemented with the sequential/parallel library preparation described herein.

### Alternative Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

### Partitioning the Sample Into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein), and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) includes DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher level of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled, and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.

(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and including a molecule including a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

### Subjecting the First Subsample to a Procedure That Affects a First Nucleobase in the DNA Differently from a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA of the first subsample includes bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase includes one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9:5068..

In some embodiments, the procedure that affects a first nucleobase in the DNA differently from a second nucleobase in the DNA of the first subsample includes oxidative bisulfite (Ox-BS) conversion. In some embodiments, the procedure that affects a first nucleobase in the DNA differently from a second nucleobase in the DNA of the first subsample includes Tet-assisted bisulfite (TAB) conversion. In some embodiments, the procedure that affects a first nucleobase in the DNA differently from a second nucleobase in the DNA of the first subsample includes Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes APOBEC-coupled epigenetic (ACE) conversion.

In some embodiments, procedure that affects a first nucleobase in the DNA differently from a second nucleobase in the DNA of the first subsample includes enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample includes separating DNA originally including the first nucleobase from DNA not originally including the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques including methylated DNA immunoprecipitation (MeDIP) can be used to separate DNA containing modified bases such as mA from other DNA. See, e.g., Kumar et al., Frontiers Genet. 2018; 9: 640; Greer et al., Cell 2015; 161: 868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### Enriching/Capturing Step, Amplification, Adaptors, Barcodes

In some embodiments, methods disclosed herein comprise a step of capturing one or more sets of target regions of DNA, such as cfDNA. Capture may be performed using any suitable approach known in the art. In some embodiments, capturing includes contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. Where the first subsample undergoes a separation step (e.g., separating DNA originally including the first nucleobase (e.g., hmC) from DNA not originally including the first nucleobase, such as hmC-seal), capturing may be performed on any, any two, or all of the DNA originally including the first nucleobase (e.g., hmC), the DNA not originally including the first nucleobase, and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein includes capturing cfDNA obtained from a test subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein includes contacting cfDNA obtained from a test subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test fsor perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein. In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions including captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed in the “General Features of the Methods” section below, which can be combined to the extent practicable with any of the foregoing embodiments and the embodiments set forth in the introduction and summary section.

### Captured Set

In some embodiments, a captured set of DNA (e.g., cfDNA) is provided. With respect to the disclosed methods, the captured set of DNA may be provided, e.g., by performing a capturing step after a partitioning step as described herein. The captured set may comprise DNA corresponding to a sequence-variable target region set, an epigenetic target region set, or a combination thereof. In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, including, respectively, DNA corresponding to a sequence-variable target region set and DNA corresponding to an epigenetic target region set. The first and second captured sets may be combined to provide a combined captured set.

In some embodiments in which a captured set including DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2-to 1.4-fold greater concentration, a 1.4-to 1.6-fold greater concentration, a 1.6-to 1.8-fold greater concentration, a 1.8-to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11 -fold greater concentration, an 11- to 12 -fold greater concentration a 12- to 13 -fold greater concentration, a 13- to 14 -fold greater concentration, a 14- to 15 -fold greater concentration, a 15- to16 -fold greater concentration, a 16- to 17 -fold greater concentration, a 17- to 18-fold greater concentration, an 18- to 19 -fold greater concentration, a 19- to 20 -fold greater concentration, a 20- to 30 -fold greater concentration, a 30- to 40 -fold greater concentration, a 40- to 50-fold greater concentration, a 50- to 60 -fold greater concentration, a 60- to 70 -fold greater concentration, a 70- to 80 -fold greater concentration, a 80- to 90 -fold greater concentration, a 90- to 100-fold greater concentration, a 10- to 20 -fold greater concentration, a 10- to 40 -fold greater concentration, a 10- to 50 -fold greater concentration, a 10- to 70 -fold greater concentration, or a 10- to 100 -fold greater concentration. The degree of difference in concentrations accounts for normalization for the footprint sizes of the target regions, as discussed in the definition section.

### Epigenetic Target Region Set

The epigenetic target region set may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein. In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### Hypermethylation Variable Target Regions

In some embodiments, the epigenetic target region set includes one or more hypermethylation variable target regions. In general, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In an example, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions. In some embodiments, hypermethylation variable target regions include one or more genomic regions, where the cfDNA molecules in those regions do not differ in methylation state in cancer subjects relative to cfDNA from healthy subjects, but the presence/increased quantity of hypermethylated cfDNA in those regions is indicative of a particular tissue type (e.g., cancer origin) and is presented as cfDNA with increased apoptosis (e.g. tumor shedding) into circulation.

Hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, the probes for the epigenetic target region set comprise probes specific for one or more hypermethylation variable target regions. The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

### Hypomethylation Variable Target Regions

Global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions, where a decrease in the level of observed methylation indicates an increased likelihood that a sample (e.g., of cfDNA) contains DNA produced by neoplastic cells, such as tumor or cancer cells. In an example, hypomethylation variable target regions can include regions that do not necessarily differ in methylation state in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation variable target regions. In some embodiments, hypomethylation variable target regions include one or more genomic regions, where the cfDNA molecules in those regions do not differ in methylation state in cancer subjects relative to cfDNA from healthy subjects, but the presence/increased quantity of hypomethylated cfDNA in those regions is indicative of a particular tissue type (e.g., cancer origin) and is presented as cfDNA with increased apoptosis (e.g. tumor shedding) into circulation.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation variable target regions overlap or comprise one or both of these regions.

In some embodiments, the probes for the epigenetic target region set comprise probes specific for one or more hypomethylation variable target regions. The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or including nucleotides 8403565-8953708 and/or 151104701-151106035 of human chromosome

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. Subjects

In some embodiments, the DNA (e.g., cfDNA) is obtained from a subject having a cancer. In some embodiments, the DNA (e.g., cfDNA) is obtained from a subject suspected of having a cancer. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1,FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### Compositions Including Captured DNA

Provided herein is a combination including first and second populations of captured DNA. The first population may comprise or be derived from DNA with a cytosine modification in a greater proportion than the second population. The first population may comprise a form of a first nucleobase originally present in the DNA with altered base pairing specificity and a second nucleobase without altered base pairing specificity, wherein the form of the first nucleobase originally present in the DNA prior to alteration of base pairing specificity is a modified or unmodified nucleobase, the second nucleobase is a modified or unmodified nucleobase different from the first nucleobase, and the form of the first nucleobase originally present in the DNA prior to alteration of base pairing specificity and the second nucleobase have the same base pairing specificity. The second population does not comprise the form of the first nucleobase originally present in the DNA with altered base pairing specificity. In some embodiments, the cytosine modification is cytosine methylation. In some embodiments, the first nucleobase is a modified or unmodified cytosine and the second nucleobase is a modified or unmodified cytosine. The first and second nucleobase may be any of those discussed herein in the Summary or with respect to subjecting the first subsample to a procedure that affects a first nucleobase in the DNA differently from a second nucleobase in the DNA of the first subsample.

In some embodiments, the first population includes a sequence tag selected from a first set of one or more sequence tags and the second population includes a sequence tag selected from a second set of one or more sequence tags, and the second set of sequence tags is different from the first set of sequence tags. The sequence tags may comprise barcodes.

In some embodiments, the first population includes protected hmC, such as glucosylated hmC. In some embodiments, the first population was subjected to any of the conversion procedures discussed herein, such as bisulfite conversion, Ox-BS conversion, TAB conversion, ACE conversion, TAP conversion, TAPSβ conversion, or CAP conversion. In some embodiments, the first population was subjected to protection of hmC followed by deamination of mC and/or C. In some embodiments of the combination, the first population includes or was derived from DNA with a cytosine modification in a greater proportion than the second population and the first population includes first and second subpopulations, and the first nucleobase is a modified or unmodified nucleobase, the second nucleobase is a modified or unmodified nucleobase different from the first nucleobase, and the first nucleobase and the second nucleobase have the same base pairing specificity. In some embodiments, the second population does not comprise the first nucleobase. In some embodiments, the first nucleobase is a modified or unmodified cytosine, and the second nucleobase is a modified or unmodified cytosine, optionally wherein the modified cytosine is mC or hmC. In some embodiments, the first nucleobase is a modified or unmodified adenine, and the second nucleobase is a modified or unmodified adenine, optionally wherein the modified adenine is mA.

In some embodiments, the first nucleobase (e.g., a modified cytosine) is biotinylated. In some embodiments, the first nucleobase (e.g., a modified cytosine) is a product of a Huisgen cycloaddition to β-6-azide-glucosyl-5-hydroxymethylcytosine that includes an affinity label (e.g., biotin).

In any of the combinations described herein, the captured DNA may comprise cfDNA. The captured DNA may have any of the features described herein concerning captured sets, including, e.g., a greater concentration of the DNA corresponding to the sequence-variable target region set (normalized for footprint size as discussed above) than of the DNA corresponding to the epigenetic target region set. In some embodiments, the DNA of the captured set includes sequence tags, which may be added to the DNA as described herein. In general, the inclusion of sequence tags results in the DNA molecules differing from their naturally occurring, untagged form.

The combination may further comprise a probe set described herein or sequencing primers, each of which may differ from naturally occurring nucleic acid molecules. For example, a probe set described herein may comprise a capture moiety, and sequencing primers may comprise a non-naturally occurring label.

### Computer Systems, Processing of Real World Evidence (RWE)

Methods of the present disclosure can be implemented using, or with the aid of, computer systems. For example, such methods may comprise: partitioning the sample into a plurality of subsamples, including a first subsample and a second subsample, wherein the first subsample includes DNA with a cytosine modification in a greater proportion than the second subsample; subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity; and sequencing DNA in the first subsample and DNA in the second subsample in a manner that distinguishes the first nucleobase from the second nucleobase in the DNA of the first subsample.

In an aspect, the present disclosure provides a non-transitory computer-readable medium including computer-executable instructions which, when executed by at least one electronic processor, perform at least a portion of a method including: collecting cfDNA from a test subject; capturing a plurality of sets of target regions from the cfDNA, wherein the plurality of target region sets includes a sequence-variable target region set and an epigenetic target region set, whereby a captured set of cfDNA molecules is produced; sequencing the captured cfDNA molecules, wherein the captured cfDNA molecules of the sequence-variable target region set are sequenced to a greater depth of sequencing than the captured cfDNA molecules of the epigenetic target region set; obtaining a plurality of sequence reads generated by a nucleic acid sequencer from sequencing the captured cfDNA molecules; mapping the plurality of sequence reads to one or more reference sequences to generate mapped sequence reads; and processing the mapped sequence reads corresponding to the sequence-variable target region set and to the epigenetic target region set to determine the likelihood that the subject has cancer.

The code can be pre-compiled and configured for use with a machine have a processer adapted to execute the code or can be compiled during runtime. The code can be supplied in a programming language that can be selected to enable the code to execute in a pre-compiled or as-compiled fashion.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### Therapies and Related Administration

In certain embodiments, the methods disclosed herein relate to identifying and administering customized therapies to patients given the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

### Kits

Also provided are kits including the compositions as described herein. The kits can be useful in performing the methods as described herein. In some embodiments, a kit includes a first reagent for partitioning a sample into a plurality of subsamples as described herein, such as any of the partitioning reagents described elsewhere herein. In some embodiments, a kit includes a second reagent for subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity (e.g., any of the reagents described elsewhere herein for converting a nucleobase such as cytosine or methylated cytosine to a different nucleobase). The kit may comprise the first and second reagents and additional elements as discussed below and/or elsewhere herein.

Kits may further comprise a plurality of oligonucleotide probes that selectively hybridize to least 5, 6, 7, 8, 9, 10, 20, 30, 40 or all genes selected from the group consisting of TLCD3B, PPP1R1B, TMEM200B, GLP1R, FJX1, ZBED4, PFKFB3, MDFI, ZNF280B, CIZ1, POU4F3, MED1, ARHGEF4, SERTM1, FRMD3, NOTUM, MBP, CLDN10, FABP3, ANXA6, PM20D2, BHLHE23, LRFN5, HOXD10, ECHDC1, GSTP1, CCKBR, MIR4500HG, CALD1, PDE4B, INSC, HLA-E, NRG1, CYYR1, MIR3185, CDH12, ANP32E, SLC7A4, LTBP4, CCDC28B, DLX4, ADCY8, SP8, ASPHD1, HOXB8, AGPS, HLF, NFIX, KCNA2, ROBO3, CCDC92B, KCNH8, PPP4R4, FOXC2, PPP2R2C, CAPG, ZNF329, IL12RB2, HOXD-AS2, SORCS1, HOXD13, SAMM50, UNC13A, CACNG8, CADPS, HIF3A, CSMD3, MIR137, DLX4, BEGAIN, PHACTR2, ZNF667, FENDRR, SRP68, TUBB2A, SLC16A12, MARCKS, C1QL1, FOXL2, ERBB2, LAYN, PDGFRA, HRK, FAM20A, LOC105377879, WNT3A, MYH11, NMNAT2, KCNIP4, TRANK1, SYT2, ZNF536, NEUROG3, LIN28A, DMRT3, MCOLN2, ITGA4, PNKY, CHRNB1, GLIS3, HAGLR, PIK3CD, DIO3OS, DUOX2, CRHR1, FAM20A, RFX4, PLXNC1, P2RX6, TLCD3B, PPP1R1B, TMEM200B, GLP1R, FJX1, ZBED4, PFKFB3, MDFI, ZNF280B, CIZ1, POU4F3, MED1, ARHGEF4, SERTM1, FRMD3, NOTUM, MBP, CLDN10, FABP3, ANXA6, PM20D2, BHLHE23, LRFN5, HOXD10, ECHDC1, GSTP1, CCKBR, MIR4500HG, CALD1, PDE4B, INSC, HLA-E, NRG1, CYYR1, MIR3185, CDH12, ANP32E, SLC7A4, LTBP4, CCDC28B, DLX4, ADCY8, SP8, ASPHD1, HOXB8, AGPS, HLF, NFIX, KCNA2, ROBO3, CCDC92B, KCNH8, PPP4R4, FOXC2, PPP2R2C, CAPG, ZNF329, IL12RB2, HOXD-AS2, SORCS1, HOXD13, SAMM50, UNC13A, CACNG8, CADPS, HIF3A, CSMD3, MIR137, DLX4, BEGAIN, PHACTR2, ZNF667, FENDRR, SRP68, TUBB2A, SLC16A12, MARCKS, C1QL1, FOXL2, ERBB2, LAYN, PDGFRA, HRK, FAM20A, LOC105377879, WNT3A, MYH11, NMNAT2, KCNIP4, TRANK1, SYT2, ZNF536, NEUROG3, LIN28A, DMRT3, MCOLN2, ITGA4, PNKY, CHRNB1, GLIS3, HAGLR, PIK3CD, DIO3OS, DUOX2, CRHR1, FAM20A, RFX4, PLXNC1, P2RX6, TLCD3B, PPP1R1B, TMEM200B, GLP1R, FJX1, ZBED4, PFKFB3, MDFI, ZNF280B, CIZ1, POU4F3, MED1, ARHGEF4, SERTM1, FRMD3, NOTUM, MBP, CLDN10, FABP3, ANXA6, PM20D2, BHLHE23, LRFN5, HOXD10, ECHDC1, GSTP1, CCKBR, MIR4500HG, CALD1, PDE4B, INSC, HLA-E, NRG1, CYYR1, MIR3185, CDH12, ANP32E, SLC7A4, LTBP4, CCDC28B, DLX4, ADCY8, SP8, ASPHD1, HOXB8, AGPS, HLF, NFIX, KCNA2, ROBO3, CCDC92B, KCNH8, PPP4R4, FOXC2, PPP2R2C, CAPG, ZNF329, IL12RB2, HOXD-AS2, SORCS1, HOXD13, SAMM50, UNC13A, CACNG8, CADPS, HIF3A, CSMD3, MIR137, DLX4, BEGAIN, PHACTR2, ZNF667, FENDRR, SRP68, TUBB2A, SLC16A12, MARCKS, C1QL1, FOXL2, ERBB2, LAYN, PDGFRA, HRK, FAM20A, LOC105377879, WNT3A, MYH11, NMNAT2, KCNIP4, TRANK1, SYT2, ZNF536, NEUROG3, LIN28A, DMRT3, MCOLN2, ITGA4, PNKY, CHRNB1, GLIS3, HAGLR, PIK3CD, DIO3OS, DUOX2, CRHR1, FAM20A, RFX4, PLXNC1, P2RX6. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes including both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adaptors having distinct molecular barcodes and identical sample barcodes. The library adaptors may not be sequencing adaptors. For example, the library adaptors do not include flow cell sequences or sequences that permit the formation of hairpin loops for sequencing. The different variations and combinations of molecular barcodes and sample barcodes are described throughout and are applicable to the kit. Further, in some cases, the adaptors are not sequencing adaptors. Additionally, the adaptors provided with the kit can also comprise sequencing adaptors. A sequencing adaptor can comprise a sequence hybridizing to one or more sequencing primers. A sequencing adaptor can further comprise a sequence hybridizing to a solid support, e.g., a flow cell sequence. For example, a sequencing adaptor can be a flow cell adaptor. The sequencing adaptors can be attached to one or both ends of a polynucleotide fragment. In some cases, the kit can comprise at least 8 different library adaptors having distinct molecular barcodes and identical sample barcodes. The library adaptors may not be sequencing adaptors. The kit can further include a sequencing adaptor having a first sequence that selectively hybridizes to the library adaptors and a second sequence that selectively hybridizes to a flow cell sequence. In another example, a sequencing adaptor can be hairpin shaped. For example, the hairpin shaped adaptor can comprise a complementary double stranded portion and a loop portion, where the double stranded portion can be attached {e.g., ligated) to a double-stranded polynucleotide. Hairpin shaped sequencing adaptors can be attached to both ends of a polynucleotide fragment to generate a circular molecule, which can be sequenced multiple times. A sequencing adaptor can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adaptor can comprise 20-30, 20-40, 30-50, 30-60, 40-60, 40-70, 50-60, 50-70, bases from end to end. In a particular example, the sequencing adaptor can comprise 20-30 bases from end to end. In another example, the sequencing adaptor can comprise 50-60 bases from end to end. A sequencing adaptor can comprise one or more barcodes. For example, a sequencing adaptor can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 21, 22, 23, 24, 25, or more (or any length as described throughout) nucleic acid bases, e.g., at least 8 bases. The barcode can be contiguous or non-contiguous sequences, as described above.

The library adaptors can be blunt ended and Y-shaped and can be less than or equal to 40 nucleic acid bases in length. Other variations of the can be found throughout and are applicable to the kit.

### Biomarkers

The disclosure provides methods of using biomarkers for the diagnosis, prognosis, and therapy selection of a subject suffering from e.g., cancer. A biomarker may be any gene or variant of a gene whose presence, mutation, deletion, substitution, copy number, or translation (i.e., to a protein) is an indicator of a disease state. Biomarkers of the present disclosure may include the presence, mutation, deletion, substitution, copy number, or translation in any one or more of EGFR, KRAS, MET, BRAF, MYC, NRAS, ERBB2, ALK, Notch, PIK3CA, APC, and SMO.

A biomarker is a genetic variant associated with one or more cancers. Biomarkers may be determined using any of several resources or methods. A biomarker may have been previously discovered or may be discovered de novo using experimental or epidemiological techniques. Detection of a biomarker may be indicative of cancer when the biomarker is highly correlated to cancer. Detection of a biomarker may be indicative of cancer when a biomarker in a region or gene occur with a frequency that is greater than a frequency for a given background population or dataset.

Publicly available resources such as scientific literature and databases may describe in detail genetic variants found to be associated with cancer. Scientific literature may describe experiments or genome-wide association studies (GWAS) associating one or more genetic variants with cancer. Databases may aggregate information gleaned from sources such as scientific literature to provide a more comprehensive resource for determining one or more biomarkers. Non-limiting examples of databases include FANTOM, GTex, GEO, Body Atlas, INSiGHT, OMIM (Online Mendelian Inheritance in Man, omim.org), cBioPortal (cbioportal.org), CIViC (Clinical Interpretations of Variants in Cancer, civic.genome.wustl.edu), DOCM (Database of Curated Mutations, docm.genome.wustl.edu), and ICGC Data Portal (dcc.icgc.org). In a further example, the COSMIC (Catalogue of Somatic Mutations in Cancer) database allows for searching of biomarkers by cancer, gene, or mutation type. Biomarkers may also be determined de novo by conducting experiments such as case control or association (e.g, genome-wide association studies) studies.

As an example, biomarkers may be determined significance testing. Here. significance testing is a statistical method used to determine if an observed effect or relationship in data is likely due to chance. For example, setting a significance level (alpha) of 0.05 indicates a 5% risk of a Type I error (i.e., concluding an effect exists when it actually doesn't). Typical steps include 1) formulating a null (no effect) and an alternative (effect exists) hypotheses, calculating a test statistic (e.g., Z-score, t-score) based on the sample data, and determining the p-value, which shows the probability of obtaining results at least as extreme as the observed ones under the null hypothesis. If the p-value is less than 0.05, the null hypothesis is rejected, suggesting the effect is statistically significant and unlikely due to chance; if greater, then there's insufficient evidence to reject the null hypothesis.

In another example, biomarkers may be determined using weights in a machine learning model.

In a machine learning model, weights (or coefficients) determine the influence of each input feature or assign the importance of each input feature on the output. They are initially set to small random values and adjusted during training to minimize the prediction error. The training process involves making predictions (forward pass), calculating the error using a loss function, and computing gradients that show how much each weight affects the error (backward pass). Using an optimization algorithm like gradient descent, weights are updated iteratively in a direction that reduces the error. This process is repeated until the model converges at which point the loss function reaches a minimum, resulting in optimal weights that improve the model's predictive accuracy.

One or more biomarkers may be detected in the sequencing panel. A biomarker may be one or more genetic variants associated with cancer. Biomarkers can be selected from single nucleotide variants (SNVs), copy number variants (CNVs), insertions or deletions (e.g., indels), gene fusions and inversions. Biomarkers may affect the level of a protein. Biomarkers may be in a promoter or enhancer, and may alter the transcription of a gene. The biomarkers may affect the transcription and/or translation efficacy of a gene. The biomarkers may affect the stability of a transcribed mRNA. The biomarker may result in a change to the amino acid sequence of a translated protein. The biomarker may affect splicing, may change the amino acid coded by a particular codon, may result in a frameshift, or may result in a premature stop codon. The biomarker may result in a conservative substitution of an amino acid. One or more biomarkers may result in a conservative substitution of an amino acid. One or more biomarkers may result in a nonconservative substitution of an amino acid.

One or more of the biomarkers may be a driver mutation. A driver mutation is a mutation that gives a selective advantage to a tumor cell in its microenvironment, through either increasing its survival or reproduction. None of the biomarkers may be a driver mutation. One or more of the biomarkers may be a passenger mutation. A passenger mutation is a mutation that has no effect on the fitness of a tumor cell but may be associated with a clonal expansion because it occurs in the same genome with a driver mutation.

The frequency of a biomarker may be as low as 0.001%. The frequency of a biomarker may be as low as 0.005%. The frequency of a biomarker may be as low as 0.01%. The frequency of a biomarker may be as low as 0.02%. The frequency of a biomarker may be as low as 0.03%. The frequency of a biomarker may be as low as 0.05%. The frequency of a biomarker may be as low as 0.1%. The frequency of a biomarker may be as low as 1%.

No single biomarker may be present in more than 50%, of subjects having the cancer. No single biomarker may be present in more than 40%, of subjects having the cancer. No single biomarker may be present in more than 30%, of subjects having the cancer. No single biomarker may be present in more than 20%, of subjects having the cancer. No single biomarker may be present in more than 10%, of subjects having the cancer. No single biomarker may be present in more than 5%, of subjects having the cancer. A single biomarker may be present in 0.001% to 50% of subjects having cancer. A single biomarker may be present in 0.01% to 50% of subjects having cancer. A single biomarker may be present in 0.01% to 30% of subjects having cancer. A single biomarker may be present in 0.01% to 20% of subjects having cancer. A single biomarker may be present in 0.01% to 10% of subjects having cancer. A single biomarker may be present in 0.1% to 10% of subjects having cancer. A single biomarker may be present in 0.1% to 5% of subjects having cancer.

Detection of a biomarker may indicate the presence of one or more cancers. Detection may indicate presence of a cancer selected from the group including ovarian cancer, pancreatic cancer, breast cancer, colorectal cancer, non-small cell lung carcinoma (e.g., squamous cell carcinoma, or adenocarcinoma) or any other cancer. Detection may indicate the presence of any cancer selected from the group including ovarian cancer, pancreatic cancer, breast cancer, colorectal cancer, non-small cell lung carcinoma (squamous cell or adenocarcinoma) or any other cancer. Detection may indicate the presence of any of a plurality of cancers selected from the group including ovarian cancer, pancreatic cancer, breast cancer, colorectal cancer and non-small cell lung carcinoma (squamous cell or adenocarcinoma), or any other cancer. Detection may indicate presence of one or more of any of the cancers mentioned in this application.

One or more cancers may exhibit a biomarker in at least one exon in the panel. One or more cancers selected from the group including ovarian cancer, pancreatic cancer, breast cancer, colorectal cancer, non-small cell lung carcinoma (squamous cell or adenocarcinoma), or any other cancer, each exhibit a biomarker in at least one exon in the panel. Each of at least 3 of the cancers may exhibit a biomarker in at least one exon in the panel. Each of at least 4 of the cancers may exhibit a biomarker in at least one exon in the panel. Each of at least 5 of the cancers may exhibit a biomarker in at least one exon in the panel. Each of at least 8 of the cancers may exhibit a biomarker in at least one exon in the panel. Each of at least 10 of the cancers may exhibit a biomarker in at least one exon in the panel. All of the cancers may exhibit a biomarker in at least one exon in the panel.

If a subject has a cancer, the subject may exhibit a biomarker in at least one exon or gene in the panel. At least 85% of subjects having a cancer may exhibit a biomarker in at least one exon or gene in the panel. At least 90%, of subjects having a cancer may exhibit a biomarker in at least one exon or gene in the panel. At least 92% of subjects having a cancer may exhibit a biomarker in at least one exon or gene in the panel. At least 95% of subjects having a cancer may exhibit a biomarker in at least one exon or gene in the panel. At least 96% of subjects having a cancer may exhibit a biomarker in at least one exon or gene in the panel. At least 97% of subjects having a cancer may exhibit a biomarker in at least one exon or gene in the panel. At least 98% of subjects having a cancer may exhibit a biomarker in at least one exon or gene in the panel. At least 99% of subjects having a cancer may exhibit a biomarker in at least one exon or gene in the panel. At least 99.5% of subjects having a cancer may exhibit a biomarker in at least one exon or gene in the panel.

If a subject has a cancer, the subject may exhibit a biomarker in at least one region in the panel. At least 85% of subjects having a cancer may exhibit a biomarker in at least one region in the panel. At least 90%, of subjects having a cancer may exhibit a biomarker in at least one region in the panel. At least 92% of subjects having a cancer may exhibit a biomarker in at least one region in the panel. At least 95% of subjects having a cancer may exhibit a biomarker in at least one region in the panel. At least 96% of subjects having a cancer may exhibit a biomarker in at least one region in the panel. At least 97% of subjects having a cancer may exhibit a biomarker in at least one region in the panel. At least 98% of subjects having a cancer may exhibit a biomarker in at least one region in the panel. At least 99% of subjects having a cancer may exhibit a biomarker in at least one region in the panel. At least 99.5% of subjects having a cancer may exhibit a biomarker in at least one region in the panel.

Detection may be performed with a high sensitivity and/or a high specificity. Sensitivity can refer to a measure of the proportion of positives that are correctly identified as such. In some cases, sensitivity refers to the percentage of all existing biomarkers that are detected. In some cases, sensitivity refers to the percentage of sick people who are correctly identified as having certain disease. Specificity can refer to a measure of the proportion of negatives that are correctly identified as such. In some cases, specificity refers to the proportion of unaltered bases which are correctly identified. In some cases, specificity refers to the percentage of healthy people who are correctly identified as not having certain disease. The non-unique tagging method described previously significantly increases specificity of detection by reducing noise generated by amplification and sequencing errors, which reduces frequency of false positives. Detection may be performed with a sensitivity of at least 95%, 97%, 98%, 99%, 99.5%, or 99.9% and/or a specificity of at least 80%, 90%, 95%, 97%, 98% or 99%. Detection may be performed with a sensitivity of at least 90%, 95%, 97%, 98%, 99%, 99.5%, 99.6%, 99.98%, 99.9% or 99.95%. Detection may be performed with a specificity of at least 90%, 95%, 97%, 98%, 99%, 99.5%, 99.6%, 99.98%, 99.9% or 99.95%. Detection may be performed with a specificity of at least 70% and a sensitivity of at least 70%, a specificity of at least 75% and a sensitivity of at least 75%, a specificity of at least 80% and a sensitivity of at least 80%, a specificity of at least 85% and a sensitivity of at least 85%, a specificity of at least 90% and a sensitivity of at least 90%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% a sensitivity of 100%. In some cases, the methods can detect a biomarker at a sensitivity of sensitivity of about 80% or greater. In some cases, the methods can detect a biomarker at a sensitivity of sensitivity of about 95% or greater. In some cases, the methods can detect a biomarker at a sensitivity of sensitivity of about 80% or greater, and a sensitivity of sensitivity of about 95% or greater.

Detection may be highly accurate. Accuracy may apply to the identification of biomarkers in cell free DNA, and/or to the diagnosis of cancer. Statistical tools, such as co-variate analysis described above, may be used to increase and/or measure accuracy. The methods can detect a biomarker at an accuracy of at least 80%, 90%, 95%, 97%, 98% or 99%, 99.5%, 99.6%, 99.98%, 99.9%, or 99.95%. In some cases, the methods can detect a biomarker at an accuracy of at least 95% or greater.

### Cancer Treatments, Therapies

It has been reported that treating colon cancer cells and tumor organoids with another derivative of a hypomethylating agent (5-aza-2′-deoxycytidine) was sufficient to induce a growth-inhibiting immune response by triggering retrotransposon expression. Combinations of DNMTi and HDACi selectively induced LTR retrotransposons more efficiently than using each drug individually. Baxevanis. “The Regulation and Immune Signature of Retrotransposons in Cancer” Cancers (Basel). 2023 Sep; 15(17): 4340. In other embodiments, the described methods and compositions can be applied to cancer therapies with a mechanism of action known to clearly impact methylation. Such therapies include HDAC inhibitors such as vorinostat, romidepsin, panobinostat, chidamide and belinostat HAT inhibitors such as inhibitors of GCN5-related N-acetyltransferases (GNATs) family, including GCN5 and p300/CBP-associating factor (PCAF) and MYST superfamily such as Garcinol, PU141, C646, Tip60 inhibitors TH1834, NU9056, and 6-alkylsalicylates, and BRD inhibitors such as I-BET 151, I-BET 762, OTX 015, MK-8628, birabresib, ipilimumab (Yervoy), a CTLA4 inhibitor applied based on PD-L1 protein expression, also tremelimumab (Imjuno); Nivolumab (Opdivo) is PD-1 inhibitor that can be utilized in combination with ipilimumab, optionally included platinum; Other PD-1 inhibitors include pembrolizumab (Keytruda), cemiplimab-rwlc (Libtayo), durvalumab (Imfinzi) which are utilized in unresectable NSCLC. Further information is found in Basudan Clin Pract. 2023 Feb; 13(1): 22-40 and Meng et al., Cell Death Dis 15, 3 (2024), each of which is fully incorporated by referenced herein.

In various embodiments, cancer treatments can include bevacizumab, cetuximab, encorafenib, ipilimumab, nivolumab, panitumumab, pembrolizumab, ramucirumab, regorafenib, tucatinib, ziv-aflibercept, adagrasib, and/or fruquintinib,

In various embodiments, cancer treatments can include trastuzumab, pertuzumab, ado-trastuzumab emtansine, neratinib, lapatinib, tucatinib, margetuximab, and/or trastuzumab deruxtecan 2. In various embodiments, cancer treatments can include abemaciclib, ado-trastuzumab emtansine, alpelisib, anastrozole, elacestrant dihydrochloride, capivasertib, everolimus, exemestane, fam-trastuzumab deruxtecan-nxki, fulvestrant, lapatinib ditosylate, letrozole/Ribociclib Succinate, margetuximab-cmkb, neratinib maleate, olaparib, palbociclib, pembrolizumab, pertuzumab, pertuzumab, trastuzumab, and/or hyaluronidase-zzxf, ribociclib, sacituzumab govitecan-hziy, talazoparib tosylate, tamoxifen citrate, toremifene, trastuzumab, and/or tucatinib. In various embodiments, cancer treatments can include abraxane (paclitaxel albumin-stabilized nanoparticle formulation), aredia (pamidronate disodium), capecitabine, cyclophosphamide, doxorubicin hydrochloride, ellence (epirubicin hydrochloride), 5-fu (fluorouracil injection), gemzar (gemcitabine hydrochloride), halaven (eribulin mesylate), infugem (gemcitabine hydrochloride), ixempra (ixabepilone), paclitaxel, taxotere (docetaxel), tecentriq (atezolizumab), tepadina (thiotepa), thiotepa, trexall (methotrexate sodium), truqap (capivasertib), vinblastine sulfate, and/or zoladex (goserelin acetate). In various embodiments, cancer treatments can include olaparib, talazoparib, rucaparib, and/or niraparib+abiraterone acetate.

In various embodiments, cancer treatments can include imatinib, gefatinib, afatinib, dacomitinib, sunitinib, sorafenib, vandetanib, brivanib, cabozantib, neratinib, tivantinib, bevacizumab, cixutumumab, dalotuzumab, figitumumab, rilotumumab, onartuzumab, ganitumab, ramucirumab, ridaforolimus, tensirolimus, everolimus, BMS-690514, BMS-754807, EMD 525797, GDC-0973, GDC-0941, MK-2206, AZD6244, GSK1120212, PX-866, XL821, IMC-A12, MM-121, PF-02341066, RG7160, and Sym004. Antibodies suitable for use as anti-EGFR therapy include cetuximab (Trade Name: Erbitux) and panitumumab (Trade Name: Vectibex). In some cases. In some cases, the cancer treatment includes EGFR tyrosine kinase inhibitors such as gefitinib (Trade Name: Iressa), erlotinib (Trade Name: Tarceva), lapatinib, canertinib, and/or cetuximab.

In other embodiments, the cancer treatment can include atezolizumab (Tecentriq), imatinib, gefatinib, afatinib, dacomitinib, sunitinib, sorafenib, vandetanib, brivanib, cabozantib, neratinib, tivantinib, bevacizumab, cixutumumab, dalotuzumab, figitumumab, rilotumumab, onartuzumab, ganitumab, ramucirumab, ridaforolimus, tensirolimus, everolimus, relatlimab, osimertinib, BMS-690514, BMS-754807, EMD 525797, GDC-0973, GDC-0941, MK-2206, AZD6244, GSK1120212, PX-866, XL821, IMC-A12, MM-121, PF-02341066, RG7160, and/or Sym004. Antibodies suitable for use as anti-EGFR therapy include cetuximab (Trade Name: Erbitux) and panitumumab (Trade Name: Vectibex). In some cases. In some cases, the cancer treatment includes EGFR tyrosine kinase inhibitors such as gefitinib (Trade Name: Iressa), erlotinib (Trade Name: Tarceva), lapatinib, canertinib, and/or cetuximab. In some instances, therapies may be used in combination, such as an anti-EGFR therapy and an anti-EGFR therapy. Anti-EGFR therapy may be used in combination with any combination of chemotherapeutic agents or chemotherapeutic regimens, for example, FOLFOX (fluorouracil [5-FU]/leucovorin/oxaliplatin), FOLFIRI (5-FU/leucovorin/irinotecan), and the like. In some aspects, a cancer treatment is administered to a subject. In some cases, the cancer treatment is administered in combination another therapy, such as a non-anti-EGFR therapy with anti-EGFR therapy. In other embodiments, the treatment may include monoclonal antibodies (mAb), multispecific antibodies, antibody-drug conjugates (ADC), cell therapy, fusion protein, drug combination, antibody fragments (Ab fragments), vaccines, and/or nanoparticles.

### Sequencing Panel

To improve the likelihood of detecting tumor indicating mutations, the region of DNA sequenced may comprise a panel of genes or genomic regions. Selection of a limited region for sequencing (e.g., a limited panel) can reduce the total sequencing needed (e.g., a total amount of nucleotides sequenced. A sequencing panel can target a plurality of different genes or regions to detect a single cancer, a set of cancers, or all cancers.

In some aspects, a panel targets a plurality of different genes or genomic regions is selected such that a determined proportion of subjects having a cancer exhibits a genetic variant or biomarker in one or more different genes or genomic regions in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity and/or a theoretical accuracy for detecting one or more genetic variants in a sample.

Probes for detecting the panel of regions can include those for detecting hotspots regions as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some embodiments, markers for a tissue of origin are tissue-specific epigenetic markers.

The one or more regions in the panel can comprise one or more loci from one or a plurality of genes. The plurality of genes may be selected for sequencing and biomarker detection. Genes included in the region to be sequenced may be selected from genes known to be involved in cancer, or from genes not involved in cancer. For example, the plurality of genes in the panel may be oncogenes, tumor suppressors, growth factors, DNA repair genes, signaling genes, transcription factors, receptors or metabolic genes. Examples of genes that may be in the panel include, but are not limited to: NEFM, SOX4, INTS1, RPSAP52,HMGA2, TMEM217,TBC1D22B, ADGRB1, AFAP1L2, LOC101927179,CORIN, MRPL4, FBXO24, BBC3, RNF167, RGMB, ZFYVE16, TRIM39,HCG18, ERICD, COPRS, MAFK, CNTNAP2, SHH, NDUFB9, LVRN, GAD2, MYL6B, NFXL1, RAD51, DNAJC25-GNG10, LINC00693, SOX4, PDE1B, MYC, KHDRBS2, RARRES2, LINC02062,CHD1, ARL13B, ZBTB18, H4C3, LOC390877, CCDC184, SUSD6, ABR, TUBGCP3, OTX1, PLEKHD1, SOX2-OT, ARL2-SNX15,ARL2, GFI1, C5orf38, LDB3, ERFE, KCNK10, TMEM145, SPTBN2, PLCG2, CYSTM1, C1orf21, HPCAL1, DGLUCY, ERICD, AGO3, RASA1, AHDC1, P2RY2, MIR9-3HG, TENT5A, FA2H, NUCKS1, SSH2, SCRN3, LOC344967,N4BP2, PALM2AKAP2, TRAF1, NUCKS1, YPEL5, HAND2-AS1, SLC25A37, WIPF3, CFAP221, RAD21-AS1,RAD21, PPFIA3, ADRA1A, KHSRP, TRIM39-RPP21,RPP21, MAPRE2, LOC344967, N4BP2, LOC115308161,ARID1B, STYX, PCDHGA2, PCDHGA12, PCDHGA9, PCDHGB7, PCDHGA3, PCDHGA10, PCDHGC3, PCDHGA5, PCDHGA6, PCDHGB4, PCDHGA7, PCDHGB6,PCDHGA1,PCDHGB1,PCDHGA4,PCDHGB2,PCDHGB3,PCDHGA11,PCDHGB, PCDHGA8, LRAT, JARID2-AS1,JARID2, TNS3, FOLH1, STX1A, H4C3, SEMA3F, SEMA3F-AS1, NDRG4, POLE, IHH, LOC388780, RPL23, CUX1, LNX2, PTPN2, RTN4RL2, SLC30A1, LOC100129917, CTD-2297D10.2, RUSC1,RUSC1-AS1, YLPM1, GRIK3, MAD1L1, RAG1, MARCHF3, NEXN-AS1,NEXN, EXD3, PYCR1, ICAM5, ERFL, SMAD7, FAM219A, CDCA7, LOC388780, LOC100505715, CD58, CAMTA2, HS3ST4, CHD3, PRNP, FAM189B, TTC9C, ITPKB, FRMD4A, DENND4B, MCIDAS, TXNL4A, IDI1, KCNH2, SHC1, LOC101928120, AGTRAP, MSC-AS1,MSC, WBP1,INO80B-WBP1, EEF1AKMT4, EEF1AKMT4-ECE2.

In some cases, the one or more regions in the panel can comprise one or more loci from one or a plurality of genes, including one or more of NEFM, SOX4, INTS1, RPSAP52,HMGA2, TMEM217,TBC1D22B, ADGRB1, AFAP1L2, LOC101927179,CORIN, MRPL4, FBXO24, BBC3, RNF167, RGMB, ZFYVE16, TRIM39,HCG18, ERICD, COPRS, MAFK, CNTNAP2, SHH, NDUFB9, LVRN, GAD2, MYL6B, NFXL1, RAD51, DNAJC25-GNG10, LINC00693, SOX4, PDE1B, MYC, KHDRBS2, RARRES2, LINC02062,CHD1, ARL13B, ZBTB18, H4C3, LOC390877, CCDC184, SUSD6, ABR, TUBGCP3, OTX1, PLEKHD1, SOX2-OT, ARL2-SNX15,ARL2, GFI1, C5orf38, LDB3, ERFE, KCNK10, TMEM145, SPTBN2, PLCG2, CYSTM1, C1orf21, HPCAL1, DGLUCY, ERICD, AGO3, RASA1, AHDC1, P2RY2, MIR9-3HG, TENT5A, FA2H, NUCKS1, SSH2, SCRN3, LOC344967,N4BP2, PALM2AKAP2, TRAF1, NUCKS1, YPEL5, HAND2-AS1, SLC25A37, WIPF3, CFAP221, RAD21-AS1,RAD21, PPFIA3, ADRA1A, KHSRP, TRIM39-RPP21,RPP21, MAPRE2, LOC344967, N4BP2, LOC115308161,ARID1B, STYX, PCDHGA2, PCDHGA12, PCDHGA9, PCDHGB7, PCDHGA3, PCDHGA10, PCDHGC3, PCDHGA5, PCDHGA6, PCDHGB4, PCDHGA7, PCDHGB6,PCDHGA1,PCDHGB1,PCDHGA4,PCDHGB2,PCDHGB3,PCDHGA11,PCDHGB, PCDHGA8, LRAT, JARID2-AS1,JARID2, TNS3, FOLH1, STX1A, H4C3, SEMA3F, SEMA3F-AS1, NDRG4, POLE, IHH, LOC388780, RPL23, CUX1, LNX2, PTPN2, RTN4RL2, SLC30A1, LOC100129917, CTD-2297D10.2, RUSC1,RUSC1-AS1, YLPM1, GRIK3, MAD1L1, RAG1, MARCHF3, NEXN-AS1,NEXN, EXD3, PYCR1, ICAM5, ERFL, SMAD7, FAM219A, CDCA7, LOC388780, LOC100505715, CD58, CAMTA2, HS3ST4, CHD3, PRNP, FAM189B, TTC9C, ITPKB, FRMD4A, DENND4B, MCIDAS, TXNL4A, IDI1, KCNH2, SHC1, LOC101928120, AGTRAP, MSC-AS1,MSC, WBP1,INO80B-WBP1, EEF1AKMT4, EEF1AKMT4-ECE2. In some embodiments, the one or more regions in the panel comprise one or more loci from one or a plurality of genes for detecting residual cancer after surgery. This detection can be earlier than is possible for existing methods of cancer detection. In some embodiments, the one or more regions in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some embodiments, the methods described herein detect cancer in high risk patients earlier than is possible for existing methods of cancer detection.

A region may be selected for inclusion in a sequencing panel based on a number of subjects with a cancer that have a biomarker in that gene or region. A region may be selected for inclusion in a sequencing panel based on prevalence of subjects with a cancer and a biomarker present in that gene. Presence of a biomarker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A databased may comprise information about regulatory elements in tumor samples. The information relating to the sequenced tumor samples may include the frequency various genetic variants and describe the genes or regions in which the genetic variants occur. The genetic variants may be biomarkers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with biomarker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or region may be selected for a panel where the frequency of a biomarker is significantly greater in sampled tumor tissue or circulating tumor DNA than found in a given background population. A combination of regions may be selected for inclusion of a panel such that at least a majority of subjects having a cancer will have a biomarker present in at least one of the regions or genes in the panel. The combination of regions may be selected based on data indicating that, for a particular cancer or set of cancers, a majority of subjects have one or more biomarkers in one or more of the selected regions. For example, to detect cancer 1, a panel including regions A, B, C, and/or D may be selected based on data indicating that 90% of subjects with cancer 1 have a biomarker in regions A, B, C, and/or D of the panel. Alternately, biomarkers may be shown to occur independently in two or more regions in subjects having a cancer such that, combined, a biomarker in the two or more regions is present in a majority of a population of subjects having a cancer. For example, to detect cancer 2, a panel including regions X, Y, and Z may be selected based on data indicating that 90% of subjects have a biomarker in one or more regions, and in 30% of such subjects a biomarker is detected only in region X, while biomarkers are detected only in regions Y and/or Z for the remainder of the subjects for whom a biomarker was detected. Biomarkers present in one or more regions previously shown to be associated with one or more cancers may be indicative of or predictive of a subject having cancer if a biomarker is detected in one or more of those regions 50% or more of the time. Computational approaches such as models employing conditional probabilities of detecting cancer given a known cancer frequency for a set of biomarkers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, bisulfate sequencing, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 regions. In some cases, the regions in the panel are selected that the size of the regions are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the regions in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the minor allele frequency for a given genetic variant. The minor allele frequency may refer to the frequency at which minor alleles (e.g., not the most common allele) occurs in a given population of nucleic acids, such as a sample. Genetic variants at a low minor allele frequency may have a relatively low frequency of presence in a sample. In some cases, the panel allows for detection of genetic variants at a minor allele frequency of at least 0.0001%, 0.001%, 0.005%, 0.01%, 0.05%, 0.1%, or 0.5%. The panel can allow for detection of genetic variants at a minor allele frequency of 0.001% or greater. The panel can allow for detection of genetic variants at a minor allele frequency of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of biomarkers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of biomarkers at a frequency in a sample as low as 1.0%. The panel can allow for detection of biomarkers at a frequency in a sample as low as 0.75%. The panel can allow for detection of biomarkers at a frequency in a sample as low as 0.5%. The panel can allow for detection of biomarkers at a frequency in a sample as low as 0.25%. The panel can allow for detection of biomarkers at a frequency in a sample as low as 0.1%. The panel can allow for detection of biomarkers at a frequency in a sample as low as 0.075%. The panel can allow for detection of biomarkers at a frequency in a sample as low as 0.05%. The panel can allow for detection of biomarkers at a frequency in a sample as low as 0.025%. The panel can allow for detection of biomarkers at a frequency in a sample as low as 0.01%. The panel can allow for detection of biomarkers at a frequency in a sample as low as 0.005%. The panel can allow for detection of biomarkers at a frequency in a sample as low as 0.001%. The panel can allow for detection of biomarkers at a frequency in a sample as low as 0.0001%. The panel can allow for detection of biomarkers in sequenced cfDNA at a frequency in a sample as low as 1.0% to 0.0001%. The panel can allow for detection of biomarkers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the regions in the panel.

The panel can comprise one or more regions from each of one or more genes. In some cases, the panel can comprise one or more regions from each of at least 1, 2, 3, 4, 5, 6, 7, 8, 9, 10, 15, 20, 25, 30, 40, 50, or 80 genes. In some cases, the panel can comprise one or more regions from each of at most 1, 2, 3, 4, 5, 6, 7, 8, 9, 10, 15, 20, 25, 30, 40, 50, or 80 genes. In some cases, the panel can comprise one or more regions from each of from about 1 to about 80, from 1 to about 50, from about 3 to about 40, from 5 to about 30, from 10 to about 20 different genes.

The regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected.

The regions in the panel can be selected so that they comprise sequences differentially transcribed across one or more tissues. In some cases, the regions can comprise sequences transcribed in certain tissues at a higher level compared to other tissues. For example, the regions can comprise sequences transcribed in certain tissues but not in other tissues.

The regions in the panel can comprise coding and/or non-coding sequences. For example, the regions in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the regions in the panel can comprise sequences in non-coding RNA, e.g., ribosomal RNA, transfer RNA, Piwi-interacting RNA, and microRNA.

The regions in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The regions in the panel can be selected to detect the cancer with a sensitivity of 100%.

The regions in the panel can be selected to detect (diagnose) a cancer with a desired level of specificity (e.g., through the detection of one or more genetic variants). For example, the regions in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a specificity of 100%.

The regions in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, regions in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The regions in the panel can be selected to detect (diagnose) a cancer with a desired accuracy. As used herein, the term “accuracy” may refer to the ability of a test to discriminate between a disease condition (e.g., cancer) and health. Accuracy may be can be quantified using measures such as sensitivity and specificity, predictive values, likelihood ratios, the area under the ROC curve, Youden's index and/or diagnostic odds ratio.

Accuracy may presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The regions in the panel can be selected to detect cancer with an accuracy of 100%.

A panel may be selected such that when one or more regions or genes in the panel are removed, specificity is appreciably decreased. Removal of one region from the panel may result in a decrease in specificity of at least 5%, 10%, 15%, 20%, 25%, 30%, 35%, 40%, 45%, 50%, or more.

A panel may be selected such that the addition of one or more regions or genes to the panel does not appreciably increase the specificity of the panel, e.g., does not increase the specificity by more than 1%, 2%, 5%, 10%, 15%, or 20%.

A panel may be of a size such that when one or more regions or genes in the panel are removed, this appreciably decreases sensitivity, e.g., sensitivity is decreased by at least 5%, 10%, 15%, 20%, 25%, 30%, 35%, 40%, 45%, 50%, or more.

A panel may be selected such that the addition of one or more regions or genes to the panel does not appreciably increase the sensitivity of the panel, e.g., does not increase the sensitivity by more than 1%, 2%, 5%, 10%, 15%, or 20%.

A panel may be of a size such that when one or more regions or genes in the panel are removed, accuracy is appreciably decreased, e.g., accuracy is decreased by at least 5%, 10%, 15%, 20%, 25%, 30%, 35%, 40%, 45%, 50%, or more.

A panel may be selected such that the addition of one or more regions or genes to the panel does not appreciably increase the accuracy of the panel, e.g., does not increase the accuracy by more than 1%, 2%, 5%, 10%, 15%, or 20%.

A panel may be of a size such that when one or more regions or genes the panel are removed, positive predictive value is appreciably decreased, e.g., positive predictive value is decreased by at least 5%, 10%, 15%, 20%, 25%, 30%, 35%, 40%, 45%, 50%, or more.

A panel may be selected such that the addition of one or more regions or genes to the panel does not appreciably increase the positive predictive value of the panel, e.g., does not increase the positive predictive value by more than 1%, 2%, 5%, 10%, 15%, or 20%

A panel may be selected to be highly sensitive and detect low frequency genetic variants. For instance, a panel may be selected such that a genetic variant or biomarker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Regions in a panel may be selected to detect a biomarker present at a frequency of 1% or less in a sample with a sensitivity of 70% or greater. A panel may be selected to detect a biomarker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a biomarker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a biomarker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or biomarker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Regions in a panel may be selected to detect a biomarker present at a frequency of 1% or less in a sample with a specificity of 70% or greater. A panel may be selected to detect a biomarker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a biomarker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a biomarker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or biomarker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Regions in a panel may be selected to detect a biomarker present at a frequency of 1% or less in a sample with an accuracy of 70% or greater. A panel may be selected to detect a biomarker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a biomarker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a biomarker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or biomarker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it should be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

### EXAMPLES

### Example 1—Methods

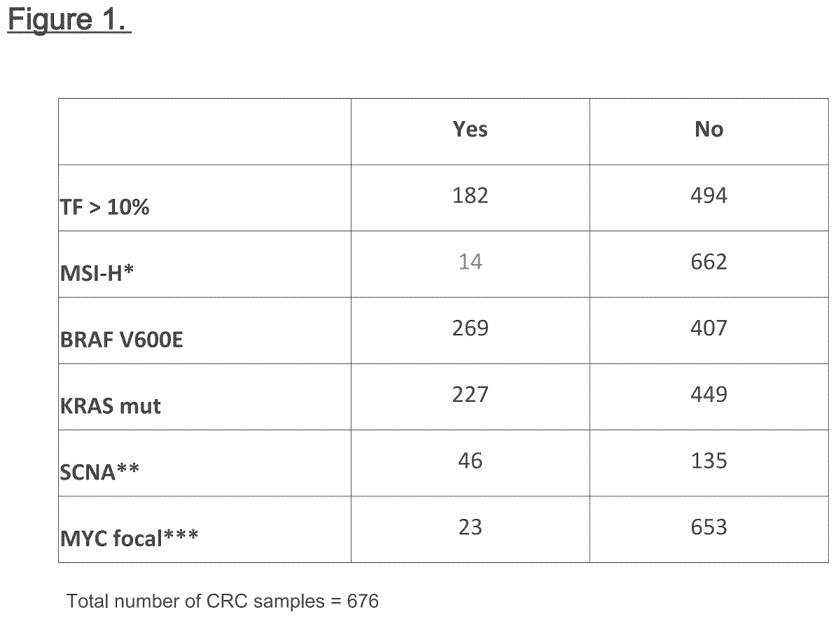

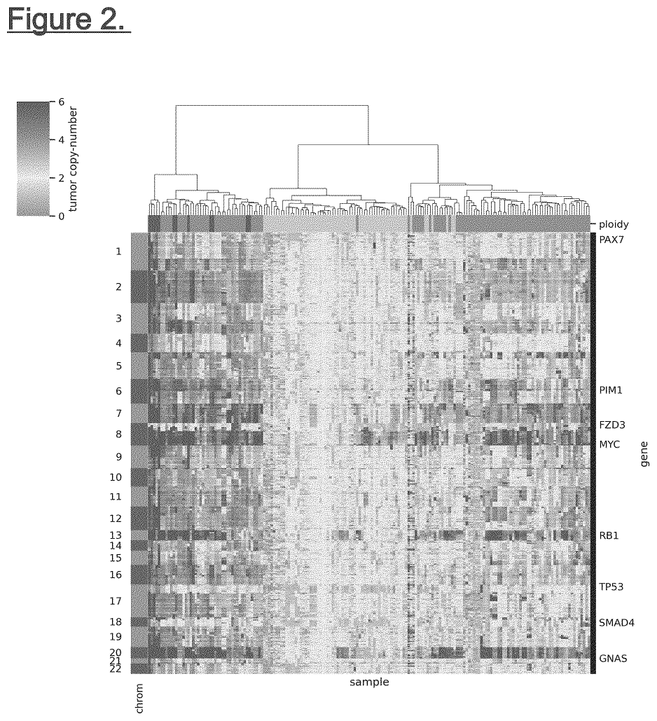

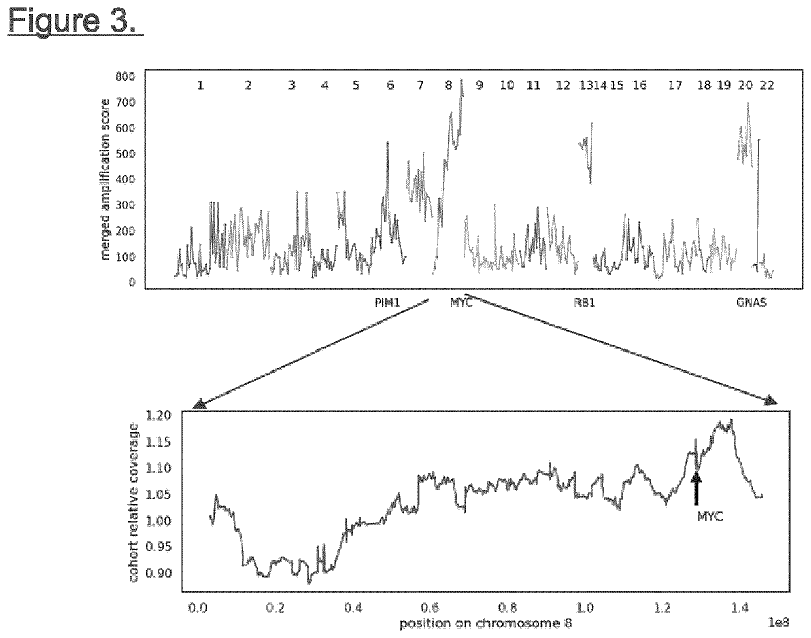

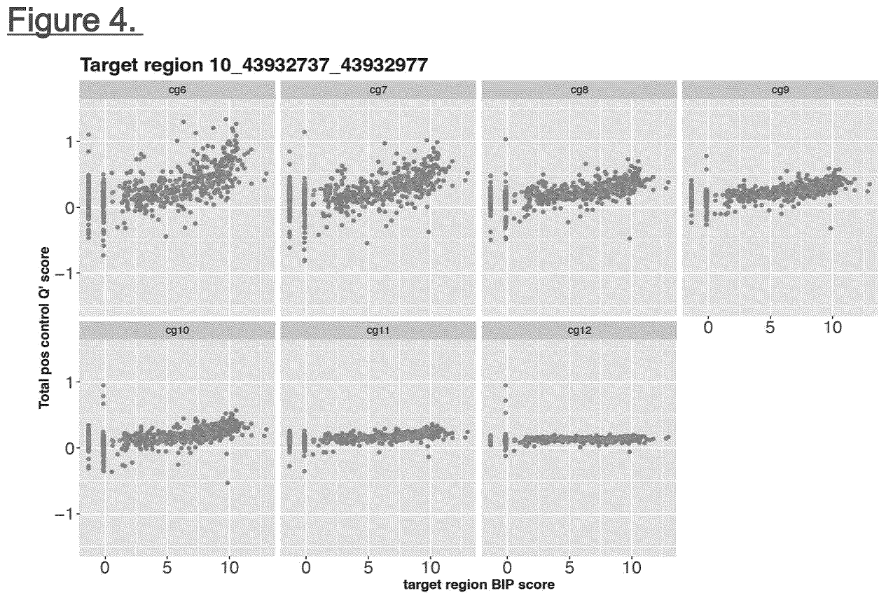

Methylation scores from a methylation-based liquid biopsy panel were generated using a training set of 676 CRC plasma samples. Features of these samples are shown in FIG. 1. Of this dataset, there were a very small number of MSI-H samples, SCNA call only was provided for samples with TF>10% as shown in FIG. 2 (cluster data based on per-gene estimated copy number status, three distinct clusters likely driven by extent of large-scale whole-genome doubling events and subsequent compensatory deletion), and no MYC activation status and very small number of focal amplification calls. For CNV data, a consistent 8p-loss, 8q-gain pattern was seen in most samples. This allows one to drill down into recurrent events at high resolution using both on-target and off-target sequencing data, providing whole genome resolution as shown in FIG. 3. Samples clustered included targeting of regions bsaed on SD, normalized score and abs (Pearson correlation with TF) as shown in FIG. 4.

### Example 2—TCGA

Genomic profiles and the status of main driver mutations known to correlate with CIMP profiles were also generated for each sample using the genomic panel portion of a methylation based panel, which reports somatic alterations in >750 genes. Tumor fraction-independent methylation regions that had significant signal were ranked according to their variability across the training set. The methylation regions that ranked in the top 100-300, depending on the cancer indication, were selected as CIMP regions to generate liquid biopsy-based CIMP profiles. Methylation beta values produced by Illumina 450K methylation microarrays from the TCGA were used to validate the CIMP-based methylation scores across the selected genomic regions of cfDNA.

### Example 3—Results

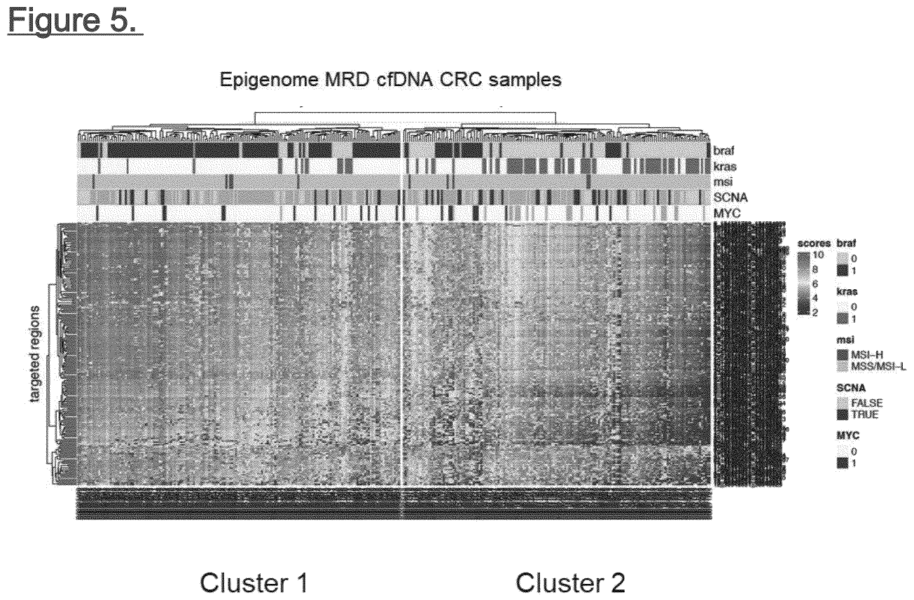

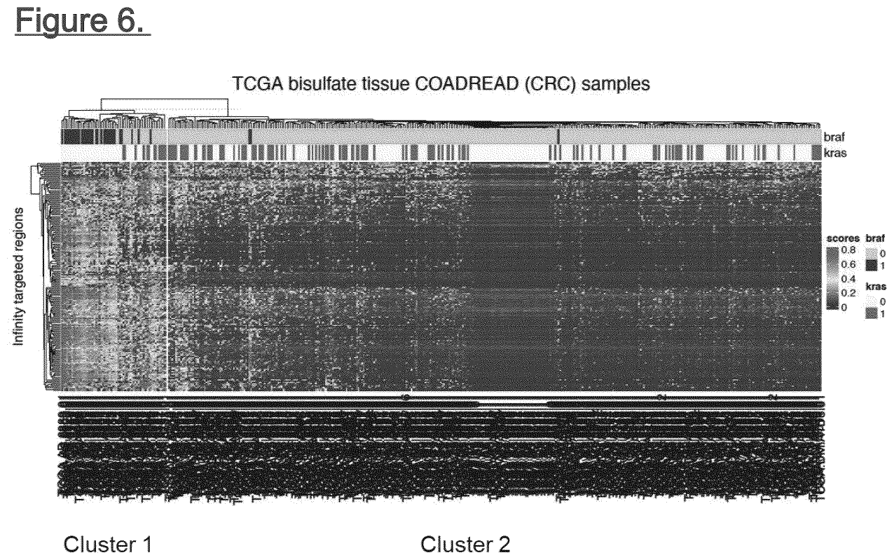

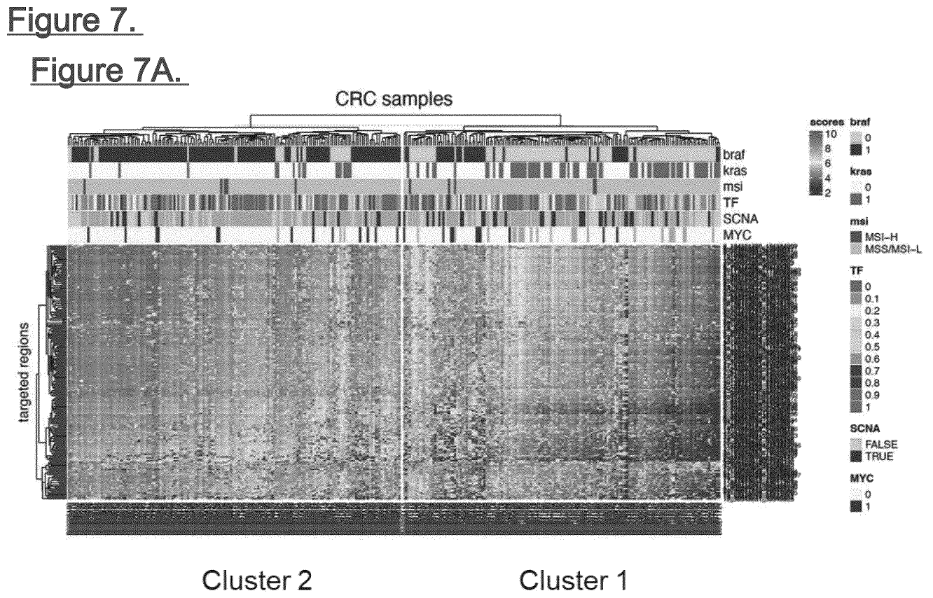

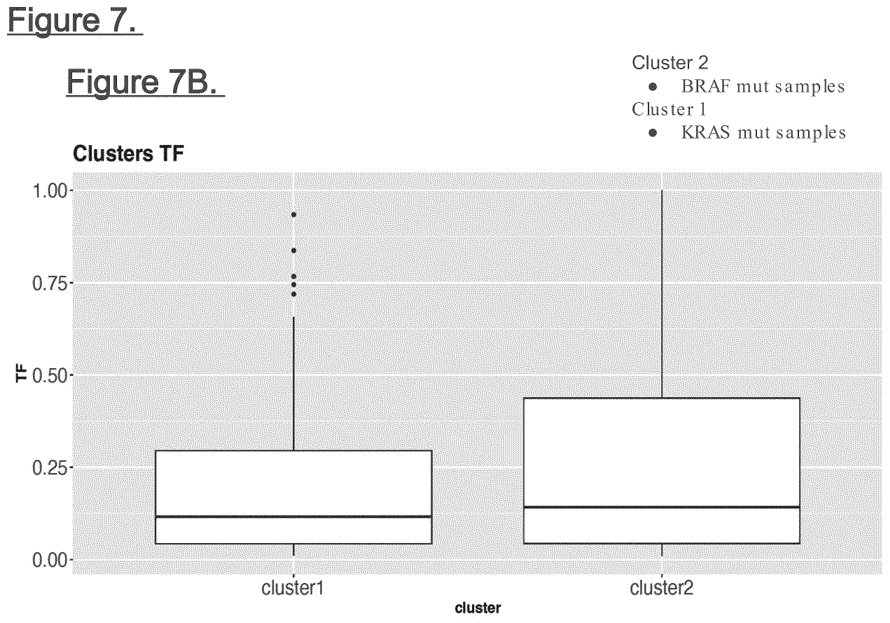

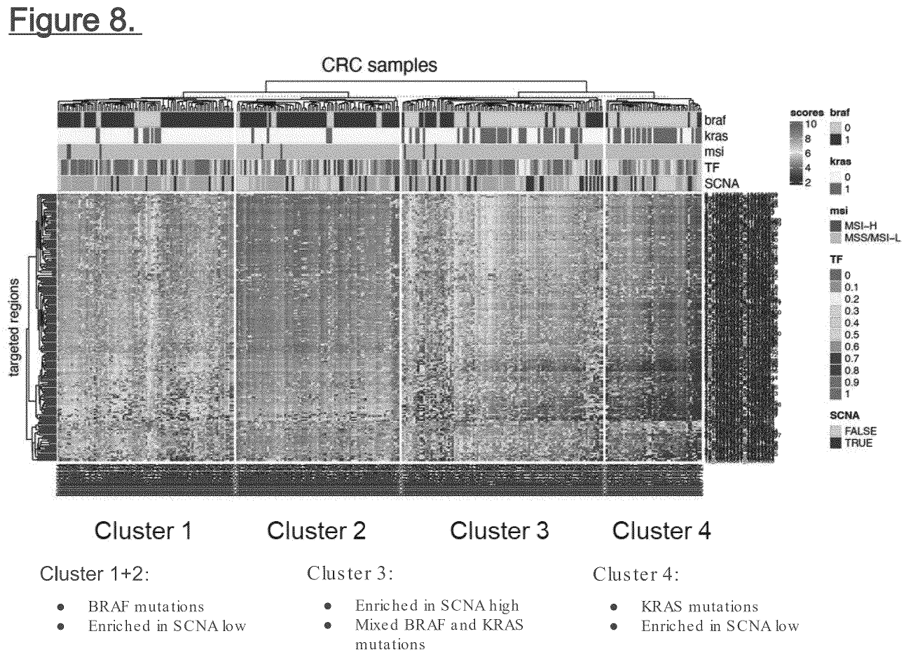

Unsupervised clustering was pursued using the methylation scores from the selected CIMP regions for each tumor indication. Specifically, normalized score of targeted regions included BIP regions scores (x-axis) correlating with CpG stratas of positive control regions (y-axis) and application of linear models to remove this correlation. This can include calculating the MBD partitioning profile for a sample and normalizing the score by regressing out the impact of MBD partitioning. Thereafter, training data such as genomic test data can be furnished to fit a linear regression model. Based on a set of training data CRC samples showed two significant CIMP profiles that correlated to BRAF and KRAS mutational status (p-values<0.0001) as shown in FIG. 5. The same selected epigenomic regions specific to each tumor type also showed hypermethylation patterns similar to tissue-based TCGA data as shown in FIG. 6. Additional clustering show BRAF and KRAS mutational status, but also enrichment in SCNA high.

### Example 4—Preliminary Findings

The results achieved herein demonstrates that a methylation-based liquid biopsy NGS test can recapitulate CIMP profiles previously observed in tissue-based methylation profiling. This allows the characterization of CIMP profiles for CRC patients to be accomplished using a convenient and non-invasive liquid biopsy approach.

### Example 5—Additional

As described in Guinney et al., four consensus molecular subtypes (CMS) with notable features may be described as CMS 1 (MSI Immune, 14%), hypermutated, microsatellite unstable, strong immune activation; CMS2 (Canonical, 37%), epithelial, chromosomally unstable, marked WNT and MYC signaling activation; CMS3 (Metabolic, 13%), epithelial, evident metabolic dysregulation; and CMS4 (Mesenchymal, 23%).

CpG island methylator phenotype (CIMP) is among unconventional pathway to CRC along with serrated polyps and have activating mutations in the BRAF proto-oncogene, frequent microsatellite instability (MSI). Fenell et al., report that the development of CIMP is critical in the progression of serrated neoplasia. Without being bound by any particular theory, it may be that CIMP can result in the silencing of key genes important for tumor progression, including the tumor-suppressor gene CDKN2A and the DNA mismatch repair gene MLH1. The relationship between CIMP and CMS subtypes is currently unclear, with neither being previously characterized by the methylation detection, characterization and classification techniques described herein.

### Example 6—Gene Enrichment Analysis

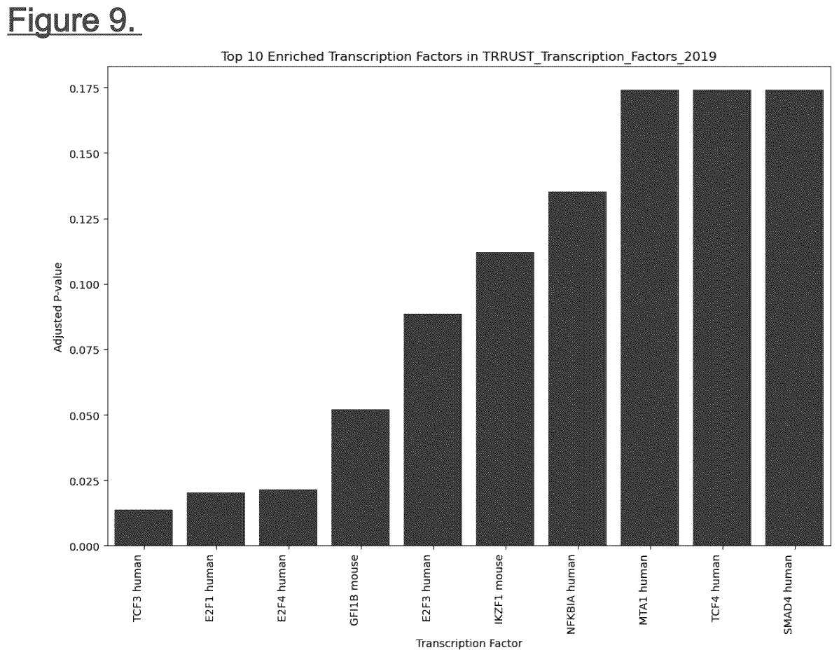

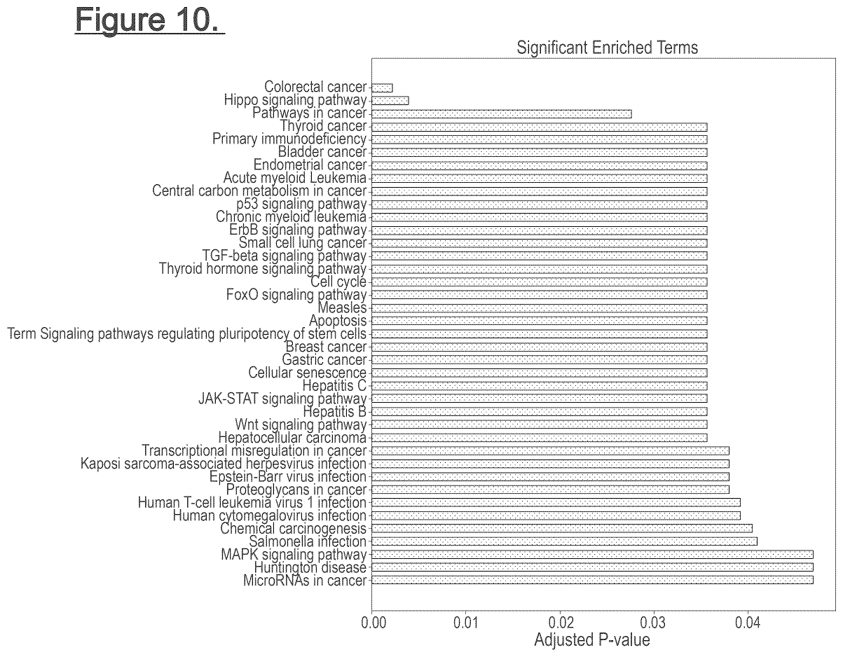

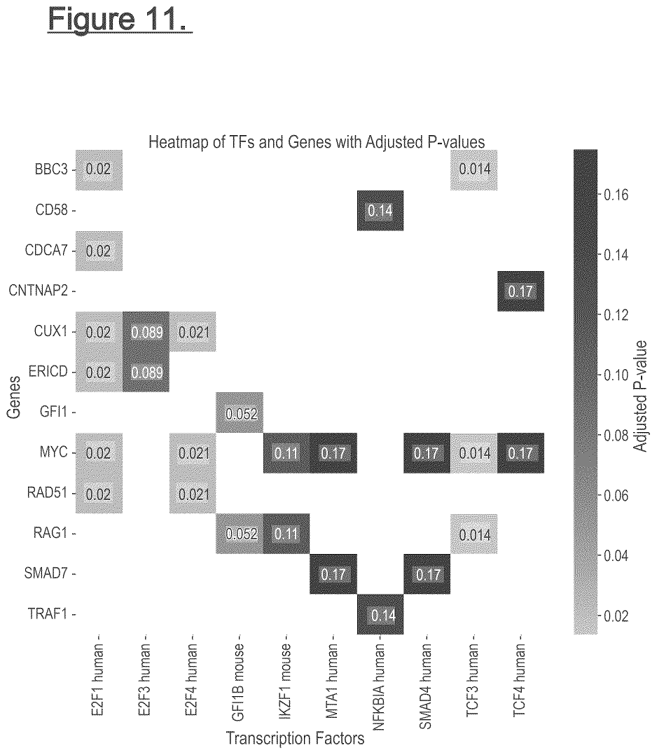

CRC subtype predictor genomic regions were used to perform gene set enrichment analyses using gp.enrichr a module within the Enrichr suite. Analyses revealed that the input gene set is significantly enriched for genes regulated by the E2F transcription factor 1(E2F1) transcription factor as shown in FIG. 9. Specifically, MYC, BBC3,RAG1 were identified as targets of E2F1, a transcription factor whose expression is increased in many human cancers. E2F 1 target genes MYC, BBC3, RAG1 are colorectal cancer genes (adjusted p-value=0.002187) with functions across multiple cancer pathways including P53, ErbB, and TGF-Beta signaling pathways.

### Example 7—Gene Pathway Analysis

Gene Set Enrichment Analysis (GSEA) using input list of E2F1 targets, including MYC, BBC3, RAG1 identified overrepresented pathways and biological processes. The analysis compared this gene list against KEGG to find significant terms. The input genes showed statistically significant enrichment CRC biological processes and function across multiple cancer pathways. The bar plot in FIG. 9 shows a visual representation of the significant enriched terms. Each bar represents a term, and its length corresponds to the adjusted p-value, indicating the significance of the enrichment.

### Example 8—E2F transcription factors

Our results suggest that the CRC predictor regions are targets of the E2F family of transcription factors including E2F1, E2F2, E2F3, E2F4, E2F5, E2F6, E2F7, and E2F8. Determining the expression changes in these transcription factors may provide additional features informative of cancer subtypes in CRC and other cancer types. These transcription factors typically form heterodimers with DP proteins (DP1 and DP2) to regulate the expression of genes necessary for cell cycle progression, DNA synthesis, DNA replication, and apoptosis.

1. A method, comprising:
detecting methylation in at least one of a plurality of sites;
generating a plurality of one or more metrics for each of the plurality of sites; and
processing the one or more metrics to characterize a sample.

2. The method of claim 1, wherein the one or more metrics are obtained from methylation calls from each of the plurality of sites.

3. The method of claim 1, comprising obtaining a sample.

4. The method of claim 1, comprising having obtained a sample.

5. The method of claim 1, further comprising building a classification model from methylation data of a set of training samples comprising one or more of: consensus molecular subtypes (CMS)1, CMS2, CMS3 and CMS4.

6. The method of claim 5, wherein the classification model is trained using clustering.

7. The method of claim 6, wherein the training comprises ranking.

8. The method of claim 7, wherein regions are selected using based on ranking.

9. The method of claim 7, wherein the ranking is based on significance and/or weights for each of the plurality of sites.

10. The method of claim 6, wherein the classification model is trained using cross-validation.

11. The method of claim 10, wherein cross-validation comprises using 1, 2, 3, 4, 5, 6, 7, 8, 9, 10-fold or more cross-validation.

12. The method of claim 1, wherein generating a plurality of one or more metrics for each of the plurality of sites comprises a linear regression, Bernoulli probability distribution and/or a mixture model.

13. The method of claim 1, wherein processing the one or more metrics to characterize a sample comprises calculating the probability of tumor molecules based on an amount of input DNA in one or more of the plurality of sites.

14. The method of claim 1, wherein the sites comprise a custom panel.

15. The method of claim 14, wherein the custom panel is configured in an in silico panel.

16. The method of claim 14, wherein the custom panel is configured in a physical panel.

17. The method of claim 14, wherein the custom panel comprises a set of oncogenes, promoter regions for a set of oncogenes, HRR genes, immuno-oncology (IO) genes, a cancer pathway, methylation peaks found in cancer or methylation peaks found in clinical samples.

18. The method of claim 14, wherein the custom panel is refined based at least on literature annotations, common methylation peak positions, and/or public datasets.

19. The method of claim 1, wherein the plurality of sites comprise one or more genes selected from the group consisting of: NEFM, SOX4, INTS1, RPSAP52,HMGA2, TMEM217,TBC1D22B, ADGRB1, AFAP1L2, LOC101927179,CORIN, MRPL4, FBXO24, BBC3, RNF167, RGMB, ZFYVE16, TRIM39,HCG18, ERICD, COPRS, MAFK, CNTNAP2, SHH, NDUFB9, LVRN, GAD2, MYL6B, NFXL1, RAD51, DNAJC25-GNG10, LINC00693, SOX4, PDE1B, MYC, KHDRBS2, RARRES2, LINC02062,CHD1, ARL13B, ZBTB18, H4C3, LOC390877, CCDC184, SUSD6, ABR, TUBGCP3, OTX1, PLEKHD1, SOX2-OT, ARL2-SNX15,ARL2, GFI1, C5orf38, LDB3, ERFE, KCNK10, TMEM145, SPTBN2, PLCG2, CYSTM1, C1orf21, HPCAL1, DGLUCY, ERICD, AGO3, RASA1, AHDC1, P2RY2, MIR9-3HG, TENT5A, FA2H, NUCKS1, SSH2, SCRN3, LOC344967,N4BP2, PALM2AKAP2, TRAF1, NUCKS1, YPEL5, HAND2-AS1, SLC25A37, WIPF3, CFAP221, RAD21-AS1,RAD21, PPFIA3, ADRA1A, KHSRP, TRIM39-RPP21,RPP21, MAPRE2, LOC344967,N4BP2, LOC115308161,ARID1B, STYX, PCDHGA2, PCDHGA12, PCDHGA9, PCDHGB7,PCDHGA3,PCDHGA10,PCDHGC3,PCDHGA5,PCDHGA6,PCDHGB4,PCDHGA 7,PCDHGB6,PCDHGA1,PCDHGB1,PCDHGA4,PCDHGB2,PCDHGB3,PCDHGA11,PCDHG B5,PCDHGA8, LRAT, JARID2-AS1,JARID2, TNS3, FOLH1, STX1A, H4C3, SEMA3F, SEMA3F-AS1, NDRG4, POLE, IHH, LOC388780, RPL23, CUX1, LNX2, PTPN2, RTN4RL2, SLC30A1, LOC100129917, CTD-2297D10.2, RUSC1,RUSC1-AS1, YLPM1, GRIK3, MAD1L1, RAG1, MARCHF3, NEXN-AS1,NEXN, EXD3, PYCR1, ICAM5, ERFL, SMAD7, FAM219A, CDCA7, LOC388780, LOC100505715, CD58, CAMTA2, HS3ST4, CHD3, PRNP, FAM189B, TTC9C, ITPKB, FRMD4A, DENND4B, MCIDAS, TXNL4A, IDI1, KCNH2, SHC1,LOC101928120, AGTRAP, MSC-AS1,MSC, WBP1,INO80B-WBP1, EEF1AKMT4,EEF1AKMT4-ECE2.

20. The method of claim 1, wherein the plurality of sites comprise one or more genes selected from the group consisting of: MYC, BBC3 and RAG1.

21. The method of claim 1, wherein characterizing the sample comprises determining gene expression of one or more biomarkers.

22. A method comprising:
detecting methylation in at least one of a plurality of sites;
generating a plurality of methylation calls for each of the plurality of sites;
obtaining one or more metrics from the methylation calls; and
processing the one or more metrics to generate a probability that a patient is afflicted with cancer.

23. The method of claim 22, wherein generating a probability that a patient is afflicted with cancer comprises determination of a cancer subtype.

24. The method of claims 23, wherein the cancer subtype is characterized as one or more of more of HR, HER2, TNBC.

25. The method of claim 22, wherein the plurality of sites comprise one or more genes selected from the group consisting of: NEFM, SOX4, INTS1, RPSAP52,HMGA2, TMEM217,TBC1D22B, ADGRB1, AFAP1L2, LOC101927179,CORIN, MRPL4, FBXO24, BBC3, RNF167, RGMB, ZFYVE16, TRIM39,HCG18, ERICD, COPRS, MAFK, CNTNAP2, SHH, NDUFB9, LVRN, GAD2, MYL6B, NFXL1, RAD51, DNAJC25-GNG10, LINC00693, SOX4, PDE1B, MYC, KHDRBS2, RARRES2, LINC02062,CHD1, ARL13B, ZBTB18, H4C3, LOC390877, CCDC184, SUSD6, ABR, TUBGCP3, OTX1, PLEKHD1, SOX2-OT, ARL2-SNX15,ARL2, GFI1, C5orf38, LDB3, ERFE, KCNK10, TMEM145, SPTBN2, PLCG2, CYSTM1, C1orf21, HPCAL1, DGLUCY, ERICD, AGO3, RASA1, AHDC1, P2RY2, MIR9-3HG, TENT5A, FA2H, NUCKS1, SSH2, SCRN3, LOC344967,N4BP2, PALM2AKAP2, TRAF1, NUCKS1, YPEL5, HAND2-AS1, SLC25A37, WIPF3, CFAP221, RAD21-AS1,RAD21, PPFIA3, ADRA1A, KHSRP, TRIM39-RPP21,RPP21, MAPRE2, LOC344967, N4BP2, LOC115308161,ARID1B, STYX, PCDHGA2, PCDHGA12, PCDHGA9, PCDHGB7,PCDHGA3,PCDHGA10,PCDHGC3,PCDHGA5,PCDHGA6, PCDHGB4, PCDHGA7, PCDHGB6, PCDHGA1, PCDHGB1, PCDHGA4, PCDHGB2, PCDHGB3,PCDHGA11,PCDHGB5,PCDHGA8, LRAT, JARID2-AS1,JARID2, TNS3, FOLH1, STX1A, H4C3, SEMA3F, SEMA3F-AS1, NDRG4, POLE, IHH, LOC388780, RPL23, CUX1, LNX2, PTPN2, RTN4RL2, SLC30A1, LOC100129917, CTD-2297D10.2, RUSC1,RUSC1-AS1, YLPM1, GRIK3, MAD1L1, RAG1, MARCHF3, NEXN-AS1,NEXN, EXD3, PYCR1, ICAM5, ERFL, SMAD7, FAM219A, CDCA7, LOC388780, LOC100505715, CD58, CAMTA2, HS3ST4, CHD3, PRNP, FAM189B, TTC9C, ITPKB, FRMD4A, DENND4B, MCIDAS, TXNL4A, IDI1, KCNH2, SHC1,LOC101928120, AGTRAP, MSC-AS1,MSC, WBP1,INO80B-WBP1, EEF1AKMT4,EEF1AKMT4-ECE2.

26. The method of claim 22, wherein the plurality of sites comprise one or more genes selected from the group consisting of: MYC, BBC3,and RAG1.

27. The method of claim 22, comprises selecting a treatment selected from Trastuzumab, Pertuzumab, Ado-trastuzumab emtansine, Neratinib, Lapatinib, Tucatinib, Margetuximab, and Trastuzumab deruxtecan.

28. The method of claim 22, comprising administering a treatment to the patient, wherein the treatment is Trastuzumab, Pertuzumab, Ado-trastuzumab emtansine, Neratinib, Lapatinib, Tucatinib, Margetuximab, and/or Trastuzumab deruxtecan.

29. The method of any preceding claim, comprising:
diagnosing a patient as being afflicted with cancer.

30. The method of any preceding claim, comprising:
prognosing a patient as susceptible to cancer.

31. The method of any one of the preceding claims, wherein the sample comprises cell-free DNA.
